SEQUENCE LISTING (13 sequences)

SEQ ID NO: 1 - DNA, 19 bp, synthetic construct
  source 1..19: mol_type = other DNA
ctgactcttatacacaagt

SEQ ID NO: 2 - DNA, 19 bp, synthetic construct
  source 1..19: mol_type = other DNA
ctgtctcttgatcagatct

SEQ ID NO: 3 - DNA, 19 bp, synthetic construct
  source 1..19: mol_type = other DNA
ctgtctcttatacacatct

SEQ ID NO: 4 - DNA, 21 bp, synthetic construct
  source 1..21: mol_type = other DNA
aagcagaagacggcatacgag

SEQ ID NO: 5 - DNA, 21 bp, synthetic construct
  source 1..21: mol_type = other DNA
acggcgaccaccgagatctac

SEQ ID NO: 6 - DNA, 21 bp, synthetic construct
  source 1..21: mol_type = other DNA
cggaagttgacagatacaggg

SEQ ID NO: 7 - DNA, 22 bp, synthetic construct
  source 1..22: mol_type = other DNA
gatcgcagaaaggtagtctcag

SEQ ID NO: 8 - DNA, 22 bp, synthetic construct
  source 1..22: mol_type = other DNA
actcagcgcaggattgtaaata

SEQ ID NO: 9 - DNA, 18 bp, synthetic construct
  source 1..18: mol_type = other DNA
cctcatgcttcggcttcc

SEQ ID NO: 10 - DNA, 20 bp, synthetic construct
  source 1..20: mol_type = other DNA
gaaacagccccaaatctcaa

SEQ ID NO: 11 - DNA, 20 bp, synthetic construct
  source 1..20: mol_type = other DNA
ttgtagccagcaagcaaatg

SEQ ID NO: 12 - DNA, 22 bp, synthetic construct
  source 1..22: mol_type = other DNA
gtggtacactgcctacagtatt

SEQ ID NO: 13 - DNA, 20 bp, synthetic construct
  source 1..20: mol_type = other DNA
gtataacgcctgggctatgt

(19) United States
(12) Patent Application Publication
(10) Pub. No.: US 2025/0382657 A1
(43) Pub. Date: Dec. 18, 2025

(54) METHODS FOR MAPPING BINDING SITES OF COMPOUNDS

(71) Applicant: Cambridge Enterprise Limited, Cambridge (GB)

(72) Inventors: Zutao Yu, Cambridge (GB); Jochen Spiegel, Cambridge (GB); Shankar Balasubramanian, Cambridge (GB)

(73) Assignee: Cambridge Enterprise Limited, Cambridge (GB)

(21) Appl. No.: 18/881,032

(22) PCT Filed: Jul. 6, 2023
(86) PCT No.: PCT/EP2023/068772

§ 371 Date: Jan. 3, 2025

(30) Foreign Application Priority Data
Jul. 7, 2022 (GB) 2209984.0
Dec. 13, 2022 (GB) 2218733.0

(51) Int. Cl. C12Q 1/6804

(52) U.S. Cl. CPC C12Q 1/6804

(57) ABSTRACT

This invention relates to mapping the binding sites of a test compound within a nucleic acid. The nucleic acid is contacted with a tagged test compound that binds to the nucleic acid or to protein associated with the nucleic acid at one or more locations. The tagged test compound is contacted with a first binding member that specifically binds to the tag and a second binding member that specifically binds to the first binding member and is attached to an activatable nuclease, such that the second binding member binds to first binding member that is bound to the tagged test compound at the one or more binding sites. The nuclease is then activated to cleave the nucleic acid at the binding sites to generate fragments. The sequence of the generated fragments is indicative of the binding sites of the test compound.

### FIELD

This invention relates to methods and kits for mapping within a nucleic acid sequence the locations of the sites at which a chemical compound binds to nucleic acid or nucleic acid associated protein.

### BACKGROUND

Small molecules that directly target DNA in cells formed the basis for the development of early anticancer and antibiotic drugs that became widely used (1). In the past two decades our understanding of genome structure and function, including the interacting chromatin proteins, has grown considerably creating many more opportunities for intervening with biology and disease states with small molecules. An essential aspect of developing small molecules probes or therapeutic drugs, is being able to validate target engagement at the molecule level (2). Where the genome itself or chromatin structure serves as the target, this necessitates mapping at the molecular level where a drug molecule binds throughout the genome.

Mapping inhibitors to chromatin has proved challenging and is mostly limited to few high affinity ligands to chromatin-binding proteins, including bromodomain inhibitor JQ1 and CDK9 inhibitor AT7519 (3-5). Genome-wide mapping involves immobilizing the small molecule using affinity tag followed by pulldown of sheared chromatin and DNA sequencing. These approaches are not applicable to many probes, as high binding affinity and low dissociating rates are needed and there is typically low signal, high background, and potential for epitope masking due to formaldehyde cross-linking. Also, the relatively low yields in DNA recovery must be overcome by large amounts of input material (4) precluding an application on rare cell populations.

Binding preferences of DNA minor groove binding molecules have been mapped biophysically using a randomised synthetic DNA oligonucleotide pool (6, 7), which does not account for differences in accessibility in native chromatin. Alternatively, the DNA binder psoralen can be UV-crosslinked to DNA and its binding sites mapped or similarly, the binding sites of a small molecule with a psoralen moiety can be mapped (3, 8). A practical challenge to overcome with conventional methods is that the strength of the non-covalent interaction needs to overcome any dissociation between the DNA and the small molecule during subsequent processing of the DNA. Moreover, DNA targeting chemotherapeutics, such as doxorubicin, have been widely used for decades in clinic. However, where and to what extent they bind in human genome has not been measurable. Thus, a general approach to map in situ small molecule-DNA interactions in intact cells would provide valuable insights into pharmacogenetics of DNA binder action and enhance our ability to exploit the genome as a therapeutic target.

### SUMMARY

The present inventors have developed a method that allows the locations of the binding sites of compounds, such as drugs, within a nucleic acid sequence, such as a genome, to be mapped efficiently and with high resolution.

A first aspect of the invention provides a method of mapping the locations of one or more binding sites of a test compound within a nucleic acid comprising;
(i) contacting the nucleic acid with a tagged test compound comprising a test compound covalently linked to a tag, wherein the tagged test compound binds to the nucleic acid or to protein associated with the nucleic acid at one or more locations within the nucleic acid,
(ii) contacting the tagged test compound with a first binding member that specifically binds to the tag, such that the first binding member binds to the tagged test compound,
(iii) contacting the nucleic acid with a second binding member that specifically binds to the first binding member and is attached to an activatable nuclease, such that the second binding member binds to first binding member that is bound to the tagged test compound at one or more binding sites,
(iv) activating the nuclease, such that the nuclease cleaves the nucleic acid at the one or more binding sites to generate fragments, and;
(v) determining the sequences of the generated fragments.

The sequences of the nucleic acid fragments may be indicative of the locations of the one or more binding sites of the test compound within the nucleic acid.

Steps (i) and (ii) of a method of the first aspect may be performed simultaneously or sequentially in any order. In some embodiments, a method of the first aspect may comprise contacting the tagged test compound with a first binding member that specifically binds to the tag, such that the first binding member binds to the tagged test compound to form a complex comprising the first binding member and the tagged test compound, and contacting the nucleic acid with the complex. In other embodiments, a method of the first aspect may comprise contacting the nucleic acid with the tagged test compound and then contacting the nucleic acid with a first binding member that specifically binds to the tag, such that the first binding member binds to the tagged test compound that is bound to the nucleic acid,

In some preferred embodiments of the first aspect, the nuclease may be a transposase. For example, a method of the first aspect may comprise;
(i) contacting the nucleic acid with a tagged test compound comprising a test compound covalently linked to a tag, wherein the tagged test compound binds to the nucleic acid or to protein associated with the nucleic acid at one or more locations within the nucleic acid,
(ii) contacting the tagged test compound with a first binding member that specifically binds to the tag, such that the first binding member binds to the tagged test compound,
(iii) contacting the nucleic acid with a second binding member that specifically binds to the first binding member and is attached to an activatable transposase that is loaded with oligonucleotide adaptors, such that the second binding member binds to first binding member that is bound to the tagged test compound at the one or more binding sites,
(iv) activating the transposase, such that the transposase cleaves the nucleic acid at the one or more binding sites to generate fragments that are labelled at each end with an oligonucleotide adaptor, and;
(v) determining the sequences of the labelled fragments.

In some embodiments of the first aspect, the sequences of the generated or labelled fragments may be determined by sequencing. For example, a method of the first aspect may comprise;
(i) contacting the nucleic acid with a tagged test compound comprising a test compound covalently linked to a tag, wherein the tagged test compound binds to the nucleic acid or to protein associated with the nucleic acid at one or more locations within the nucleic acid,
(ii) contacting the tagged test compound with a first binding member that specifically binds to the tag, such that the first binding member binds to the tagged test compound,
(iii) contacting the nucleic acid with a second binding member that specifically binds to the first binding member and is attached to an activatable transposase that is loaded with sequencing adaptors, such that the second binding member binds to first binding member that is bound to the tagged test compound at the one or more binding sites,
(iv) activating the transposase, such that the transposase cleaves the nucleic acid at the one or more binding sites to generate fragments that are labelled at each end with a sequencing adaptor, and;
(v) sequencing the labelled fragments.

In other embodiments of the first aspect, the sequences of the generated or labelled fragments may be determined by hybridisation-based techniques, preferably sequence-specific amplification.

The test compound may bind to the nucleic acid or protein associated with the nucleic acid at multiple locations within the nucleic acid. Methods of the first aspect may be useful in identifying or mapping these multiple locations.

Methods of the first aspect may comprise mapping the locations of the binding sites of multiple test compounds. For example, a method of mapping the locations of the binding sites of a population of test compounds within a nucleic acid may comprise;
(i) contacting the nucleic acid with a population of tagged test compounds, each tagged test compound in the population comprising a test compound covalently linked to a tag, wherein the tagged test compounds in the population bind to the nucleic acid or to protein associated with the nucleic acid at one or more sites within the nucleic acid,
(ii) contacting the tagged test compounds with a population of first binding members, such that each first binding member in the population specifically binds to a different tagged test compound in the population,
(iii) contacting the nucleic acid with a population of second binding members attached to activatable nucleases, such that each second binding member in the population specifically binds to a different first binding member that is bound to a tagged test compound at a binding site in the nucleic acid,
(iv) activating the nucleases, such that the nucleases cleave the nucleic acid at the one or more binding sites to generate fragments, and;
(v) determining the sequences of the generated fragments.

The sequences of nucleic acid fragments generated by a nuclease that is bound via the second and first binding members to a tagged test compound in the population may be indicative of the locations of one or more binding sites of the test compound within the nucleic acid.

Methods of the first aspect may comprise mapping the locations of the binding sites of a test compound within a first nucleic acid and a second nucleic acid having the same or different nucleotide sequences and identifying locations that are present in the first nucleic acid and not in the second nucleic acid or present in the second nucleic acid and not in the first nucleic acid. One of the first and second nucleic acids may have been subjected to a treatment. The effect of the treatment on the locations of the binding sites may be determined.

In some embodiments of the first aspect, the effect of an untagged second test compound on the locations of the one or more binding sites of the test compound within the nucleic acid; or the binding of the test compound to the one or more binding sites may be determined. For example, step (i) of the method may further comprise contacting the nucleic acid with an untagged second test compound, optionally wherein the untagged second test compound binds to the nucleic acid or to protein associated with the nucleic acid at one or more locations within the nucleic acid.

A second aspect of the invention provides a kit for mapping the locations of one or more binding sites of a test compound within a nucleic acid; the kit comprising;
a tag covalently linked or linkable to a test compound,
a first binding member that specifically binds to the tag,
a second binding member that specifically binds to the first antibody; and
a nuclease that is attached or attachable to the second binding member.

Other aspects and embodiments of the invention are described in more detail below.

### BRIEF DESCRIPTION OF THE FIGURES

FIG. 1 shows that Chemmap method reveals genomic binding sites for the BET bromodomain-targeting drug JQ1. (A) Chemmap experiment workflow: In permeabilised cells, the precomplex of biotinylated drugs and Anti-biotin antibody bind to the chromatin target (protein or DNA structure). Then a secondary antibody tethering of pA-Tn5 transposomes was recruited to the drug binding sites. Addition of Mg²⁺ activates the transposomes and integrates adapters at the drug binding sites. After DNA purification, genomic fragments with adapters at both ends are enriched via PCR, which allows for the genome-wide identification of drug binding sites by next-generation sequencing. (B) Chemical structure of biotinylated JQ1 conjugate. (C) Pairwise intersection reveals high overall consistency of enriched peaks across five technical replicates of JQ1-bio Chemmap in K562 cells. (D) Venn diagram illustrating the high confidence binding sites of JQ1 (Chemmap, top) and its protein target BRD4 (CUT&Tag, bottom) in K562 cells, among five technical and two biological replicates. There are 19,693 consensus peaks (93%) for both JQ1 and BRD4. (E) Gene browser views of JQ1 Chemmap (first track; Biotin Chemmap, second track) and BRD4 CUT&Tag (third track; no-1^{st} AB CUT&Tag as control, fourth track) compared to published JQ1 Chem-Seq (fifth track; Input Click-Chem-seq, sixth track) and BRD4 ChIP-seq (seventh track; Input ChIP-seq, eighth track) data at the CCND2 gene locus. Boxes highlight regions of respective close-up views at the scales of 50 kb and 5 kb, respectively. (F) Fraction of reads in peak (FRiP) analysis comparing JQ1 Chemmap, BRD4 CUT&Tag, JQ1 Click-Chem-seq (different JQ1 derivatives JQ1-TCO and JQ1-PA) and BRD4 ChIP-seq in K562 cells. (G) Comparison of JQ1

Chemmap and published JQ1 Chem-seq signal averaged at high confidence loci detected with BRD4 CUT&Tag in K562 cells. Two JQ1 Chemmap are highly similar curve which almost merged together, as shown in high peak with maximum peak height of 8. JQ1-TCO Click-seq and JQ1-PA Click-seq are close to the baseline. The data is normalised by sequencing depth.

FIG. 2 shows the characterisation of Chemmap for JQ1. (A) Hierarchical clustering of the Spearman correlation matrix for two biological replicates (B1 and B2) of JQ1 Chemmap in K562 cells with each 5 technical replicates (t1 to t5). (B) Pairwise intersection of enriched peaks from JQ1 Chem-map in K562 cells for a 2^{nd} biological replicate comprising five technical replicates. (C) Principal components analysis (PCA) comparing JQ1 Chemmap, Biotin Chemmap, and BRD4 CUT&Tag in K562 cells. (D) Venn diagram illustrating the high-confidence binding sites of JQ1 (Chem-map, left) and its protein target BRD4 (CUT&Tag, right) in U2OS cells. There are 9001 consensus peaks (93%) for both JQ1 and BRD4.

FIG. 3 shows genome-wide binding sites of DNA G-quadruplex binders. (A) Chemical structure of biotinylated G4 ligands PDS (left) and PhenDC3 (right). (B) Gene browser views of Chemmap for PDS-bio (first track) and PhenDC3-bio (second track) compared to CUT&Tag data for the G4 antibody BG4 (fourth track) at the KRAS locus. Sites that fold into G4 structures in vitro (called observed quadruplex sequences, OQS) are highlighted (OQS+ is plus strand, fifth track; OQS− is in reverse strand, sixth track). The box highlights the regions of a close-up view at a scale of 5 kb. (C) Venn diagrams illustrating the overlap of binding sites for the G4 ligands PDS-bio (left) and OQS (right). (D) Hierarchical clustering of the Spearman correlation matrix for PDS, PhenDC3 and BG4. (E) Venn diagrams illustrating the overlap of binding sites for the G4 ligands PDS-bio (upper left) and PhenDC3-bio (bottom) and BG4 CUT&Tag (upper right).

FIG. 4 shows the characterization of Chemmap for G4 ligands (PDS and PhenDC). (A) FRET-melting assay of G4 ligand binding to G4-forming oligonucleotides (c-Kit in left, Myc in middle, and Telo in right) and negative control dsDNA with inducing thermal stabilization (ΔTₘ). Four compounds, PDS, PDS-bio, PhenDC3, and PhenDC3-bio, are evaluated ΔTₘ of four probes at 1 μM on dsDNA are 0. Mean is represented from two independent experiments (n=2). (B) Venn diagrams illustrating the overlap of binding sites for the G4 ligands PhenDC3-bio (left) and OQS (right). (C) Principal components analysis (PCA) comparing Chemmap experiments with G4 ligands and biotin control in K562 cells. (D) Differential binding analysis for PDS Chemmap and PhenDC3 Chemmap.

FIG. 5 shows genomic binding sites of doxorubicin identified by Chemmap. (A) Chemical structure of biotinylated derivatives of Doxorubicin, Dox-bio1 (left) and Dox-bio2 (right). (B) Microscopy analysis of U2OS cells visualising nuclear enrichment of Doxorubicin (up), Dox-bio1 (middle), and Dox-bio2 (bottom). Three imaging channels: Hoechst for nuclei (left), RFP for doxorubicin and its derivatives (middle), and merging mode of Hoechst/RFP (right). (C) Principal components analysis (PCA) comparing Chemmap of different small molecules, JQ1-bio, PhenDC3-bio, Dox-bio1, and PDS-bio, in K562 cells. (D) Venn diagram showing the overlap of Dox-bio1 binding sites (left) with open chromatin mapped by ATAC-seq (right). There are 12,896 consensus peaks (95%) for both Dox-bio1 and ATAC-seq. (E) Gene browser views of Dox-bio1 Chemmap binding sites (first track) compared to a biotin control (second track) and ATAC-seq (third track). Biotin Chemmap is a control (second track).

FIG. 6 shows the characterisation of Chemmap datasets with different small molecules. (A) Tapestation analysis comparing relative DNA recovery for Dox-bio1, JQ1-bio, and Dox-bio2 using Chemmap. (B) Enrichment over random (n=1000 permutations) of JQ1-bio, PDS-bio1, PDS-bio2, PhenDC3-bio1, PhenDC3-bio2, Doxorubicin-bio1 and Doxorubicin-bio2 at genomic features from the reference human annotation GENECODE v.28. UTR untranslated region; Promoter defined as 1 kb upstream transcription start sites. Peak distribution of Chemmap experiment with JQ1-bio1, PDS-bio1, PDS-bio2, PhenDC3-bio1, PhenDC3-bio2, doxorubicin-bio1 and doxorubicin-bio2 across different genomic features. (C) FRiP analysis of Chemmap of two biological replicates with different small molecules, JQ1-bio1, PDS-bio1, PDS-bio2, PhenDC3-bio1, PhenDC3-bio2, doxorubicin-bio1 in K562 cells. (D) Gene browser views of peak profiles among JQ1bio Chemmap (first track), BRD4 CUT&Tag (second track), PDS-bio Chemmap (third track), PhenDC3-bio Chemmap (fourth track), BG4 CUT&Tag (fifth track), and doxorubicin-bio Chemmap (sixth track). (E) Differential binding analysis (FDR<0.05) for Chemmap comparing binding sites for different small molecules in K562 cells. Light grey dots represent sites where the binding is significantly different for two molecules (considering two biological replicates each with five technical replicates).

FIG. 7 shows response to drug combinations characterised by Chemmap. (A) Differential binding analysis showing differences in Dox-bio1 Chemmap peaks for Tucidinostat-treated (1 μM, 72 h) and vehicle-treated (0.1% DMSO, 72 h) K562 cells. Light grey dots represent sites where the binding is significantly different (FDR<0.05) for two molecules (considering two biological replicates, each with five technical replicates). A positive fold-change indicates an increase in Dox-bio1 binding. (B) Venn diagram illustrating the overlap of high confidence Dox-bio1 binding sites (upper right) in vehicle-treated and Tucidinostat-treated (bottom right) with ATAC-seq (upper left and bottom left) in K562 cells. (C) Enrichment over random (n=1000 permutations) of Dox-bio1 Chemmap peaks at genomic features from the reference human annotation GENECODE v.28. Up/Down, significantly up-or down-regulated peaks in Tucidinostat treated K562 cells. UTR untranslated region; Promoter defined as 1 kb upstream transcription start sites. (C) Gene browser views displaying difference in Dox-Bio1 binding for Tucidinostat-treated (first track) or vehicle-treated (second track) cells in K562 cells compared to a non-antibody control (third track) and ATAC-seq (fourth track).

FIG. 8 shows an example of workflow for a competitive Chemmap assay.

FIG. 9 shows Venn diagrams illustrating the overlap of PDS Chemmap high-confidence peaks from K562 cells dosed with unmodified PDS (4 μM for 3 hr, present in 3/5 technical replicates) (left panel) and overlap of those peaks with OQS (right panel).

FIG. 10 shows FRiP analysis comparing untreated and treated (unmodified PDS, 4 μM for 3 hr) PDS Chemmap in K562 cells.

FIG. 11 shows genome-wide mapping of biotinylated doxorubicin using pA-Tn5 and pA-MNase. Doxorubicin mapping with pA-Tn5 and MNase show similar peak distribution, such as peak 1, 2, and 3 as highlighted in the dashed box. Lane 1 is Dox Chemmap and lane 2 is Dox mapping using pA-MNase.

FIG. 12 shows the chemical structures of PIP1-bio, PIP2-bio, and PIP3-bio.

FIG. 13 shows genome-wide mapping of three biotinylated PIPs. The results also compared with biotinylated doxorubicin and genome accessible region using ATAC-seq. All three PIPs have binding to the open chromatin as exemplified by ATAC-seq and Dox-bio1 Chemmap, as shown in Peak 1. Meanwhile, PIP1-bio and PIP2-bio can also access heterochromatin and enriched at WGCWGCW region, as shown in Peak 2. Lane 1: PIP1-bio Chemmap; lane 2: PIP2-bio Chemmap; lane 3: PIP3-bio Chemmap; lane 4: Dox-bio1 Chem-map; lane 5: ATAC-seq in K562 cells; lane 6: biotin Chemmap as negative control; lane 7: motif search for WGCWGCW, W stands for A/T.

### DETAILED DESCRIPTION

This invention relates to methods and kits for mapping the locations of sites within a nucleic acid at which a test compound, such as a chemotherapeutic drug, binds to the nucleic acid or protein with the nucleic acid (i.e. nucleic acid associated protein). A nucleic acid is contacted with a test compound covalently linked to a tag (i.e. a tagged test compound). The tagged test compound binds to the nucleic acid or to protein associated with the nucleic acid at one or more locations within the nucleic acid. The nucleic acid is also contacted with a first or primary binding member that specifically binds to the tag, such that the first binding member binds to the tagged test compound. The nucleic acid is then contacted with a second or secondary binding member that specifically binds to the first binding member. The second binding member binds to the first binding member that is bound to the tagged test compound at the one or more binding sites. The nuclease attached to the second binding member is then activated to cleave the nucleic acid to generate nucleic acid fragments that contain the one or more binding sites. The sequences of the generated nucleic acid fragments may then be determined, for example by sequencing or site-specific amplification. The locations of the one or more binding sites within the nucleic acid in the sample may be identified or mapped from the sequences of the generated fragments.

Nucleic acid may be RNA. For example, the locations of the binding sites of the tagged test compound within a population of RNA molecules, such a cell transcriptome or a portion or fraction thereof may be determined. Suitable RNA molecules may include nuclear RNA molecules, such as pre-mRNA and miRNA.

Nucleic acid may be DNA. For example, the locations of the binding sites of the tagged test compound within a cell genome or a portion or fraction thereof may be determined. In some embodiments, the nucleic acid may be a population of DNA molecules, such as products generated by amplification of all or part of a genome, for example one or more regions or loci of interest.

In some embodiments, the nucleic acid may be in the form of chromatin. Chromatin is a complex of DNA and proteins that forms the chromosomes of eukaryotic cells. Chromatin for use as described herein may include part of a chromosome, a whole chromosome or more than one chromosome. In preferred embodiments, chromatin for use as described herein may include part or all of the nuclear and/or mitochondrial genome of a eukaryotic cell, for example, preferably whole genome of a eukaryotic cell. Chromatin may include heterochromatin and euchromatin.

In some embodiments, the nucleic acid may be within a sample. Suitable samples may comprise tissue, organoids, cells, cell extracts or cell fractions, for example cell organelles, such as nuclei or mitochondria.

In some preferred embodiments, the nucleic acid may be within a cell or cell extract. For example, a sample comprising one or more cells may be contacted with a tagged test compound as described herein, such that the tagged test compound contacts nucleic acid in the cells. In some embodiments, the nucleic acid may be within a single cell or an extract from a single cell. In other embodiments, the nucleic acid may be within a population of cells or an extract from a population of cells.

This may allow the locations of the binding sites of the test compound within the genome or transcriptome of the cell to be mapped. For example, a method of mapping the locations of one or more binding sites of a test compound within a cell genome or transcriptome may comprise;
(i) contacting a cell with a tagged test compound comprising a test compound covalently linked to a tag, wherein the tagged test compound binds to nucleic acid or to protein associated with nucleic acid at one or more locations within the genome or transcriptome of the cell,
(ii) contacting the tagged test compound with a first binding member that specifically binds to the tag, such that the first binding member binds to the tagged test compound,
(iii) contacting the cell with a second binding member that specifically binds to the first binding member and is attached to an activatable nuclease, such that the second binding member binds to first binding member that is bound to the tagged test compound at the one or more binding sites,
(iv) activating the nuclease, such that the nuclease cleaves the nucleic acid at the one or more binding sites to generate fragments, and;
(v) determining the sequence of the generated fragments.

The cell may be permeabilised before after or during step (i).

The sequences of the nucleic acid fragments may be indicative of the locations of the one or more binding sites of the test compound within the cell genome or transcriptome.

In some embodiments, the cell may be a prokaryotic cell. For example, a prokaryotic cell may be contacted as described herein with an anti-microbial agent covalently linked to a tag. Suitable antimicrobial agents include compounds that bind to nucleic acid-associated proteins, such as DNA gyrase.

In other embodiments, the cell may be a eukaryotic cell. Eukaryotic cells may be isolated, for example as immortalised cell lines or primary cells obtained from an individual or may be in the form of tissues or organoids. For example, the eukaryotic cells may be within a sample obtained from an individual, such as a biopsy or xenograft sample.

Suitable eukaryotic cells may include mammalian cells, preferably human cells. For example, eukaryotic cells may include somatic and germ-line cells and may be at any stage of development, including fully or partially differentiated cells or non-differentiated or pluripotent cells, including stem cells, such as adult or somatic stem cells, foetal stem cells or embryonic stem cells. Suitable eukaryotic cells also include induced pluripotent stem cells (iPSCs), which may be derived from any type of somatic cell in accordance with standard techniques. Eukaryotic cells may also include neural cells, including neurons and glial cells; contractile muscle cells; smooth muscle cells; liver cells; hormone synthesising cells; sebaceous cells; pancreatic islet cells; adrenal cortex cells; fibroblasts; mesenchymal cells; epithelial cells; keratinocytes; endothelial cells; urothelial cells; osteocytes; chondrocytes; immune cells; such as leukocytes; mesothelial cells and adipocytes.

Suitable eukaryotic cells also include normal cells or cells associated with disease conditions, for example cancer cells, such as carcinoma, sarcoma, lymphoma, blastoma or germ-line tumour cells, and cells with the genotype of a genetic disorder, such as Huntington's disease, cystic fibrosis, sickle cell disease, phenylketonuria, Down syndrome or Marfan syndrome.

A cell may be permeabilised to allow the first and second antibodies to enter and access nucleic acid inside the cell. Suitable methods for permeabilising cells are well known in the art and include contacting the cells with a detergent, such as 2-[4-(2,4,4-trimethylpentan-2-yl)phenoxy]ethanol (e.g. triton X-100™), nonyl phenoxypolyethoxylethanol (e.g. NP-40™), polyoxyethylene sorbitan monolaurate (e.g. Tween™), saponin, or digitonin. For example, cells may be permeabilised by exposure to digitonin, for example 0.05% digitonin.

In some embodiments, the cell may be a viable or live cell. For example, step (i) of a method described herein may comprise contacting a viable cell with a tagged test compound. The viable cell may be treated with or exposed to the tagged test compound, for example in a culture medium, for a defined time period, for example 1 to 48 hours, 2 to 36 hours or 3 to 24 hours, such that the tagged test compound binds to nucleic acid or nucleic acid associated protein in the cell. Following treatment with the tagged test compound, the cell may be permeabilised before contact with the first binding member in step (ii).

In some embodiments, nucleic acid or cells containing nucleic acid may be fixed before contact with the test compound. Suitable methods for fixing cells are well known in the art and include contacting the cells with an aldehyde fixative such as formaldehyde, formalin, or glutaraldehyde; or an alcohol fixative, such as methanol, ethanol, or acetone. For example, cells may be fixed by exposure to 0.1% formaldehyde.

In some embodiments, nucleic acid or cells containing nucleic acid may be in solution in methods described herein. For example, nucleic acid or cells containing nucleic acid may be contacted with the tagged test compound, first binding member and/or second binding member in solution. The nucleic acid or cells containing nucleic acid may be washed, for example by centrifugation and resuspension between steps.

In other embodiments, nucleic acid or cells containing nucleic acid may be immobilised on a solid support. A solid support is an insoluble, non-gelatinous body which presents a surface on which a capture molecule can be immobilised for capture of the eukaryotic cell. Examples of suitable supports include glass slides, microwells, membranes, or microbeads. The support may be in particulate or solid form, including for example a plate, a test tube, bead, a ball, filter, fabric, polymer or a membrane. Capture molecules may bind to proteins, glycoproteins or other molecules on the surface of the eukaryotic cell. Suitable capture molecules for eukaryotic cells are well-known in the art and include lectins that bind to extracellular glycoproteins on the cell, such as concanavalin A. In preferred embodiments, the solid support is a magnetic bead, for example a magnetic bead coated with a lectin, such as concanavalin A.

The sequence of the nucleic acid fragments may be indicative of the sequence of the nucleic acid at the location of the binding site of the test compound. In some embodiments, the test compound may bind to nucleic acid or nucleic acid associated protein at multiple locations within the nucleic acid. The sequence of the nucleic acid fragments may be indicative of the sequence of the nucleic acid at the locations of binding sites of the test compound.

The test compound may be a compound or molecule that binds to nucleic acid or to nucleic acid associated protein.

In some embodiments, the test compound may bind to nucleic acid, such as RNA or DNA. The nucleic acid may be present in or extracted from a cell, organelle or tissue; or may be a product of amplification of one or more regions or loci of interest in the nucleic acid.

In some embodiments, the test compound may bind to DNA i.e. the test compound may be a DNA-binding compound. Suitable DNA-binding compounds may include compounds that intercalate between base-pairs of chromatin DNA, compounds that bind to the major or minor groove of chromatin DNA; compounds that bind to specific secondary structures of chromatin DNA, such as G-quadruplexes, Z-DNA, H-DNA, i-motifs, and higher order structures, such as looping interactions between enhancers and promotors; and compounds that bind to other nucleic acid features, such as repetitive elements, DNA mismatch, and DNA damage sites. In other embodiments, the test compound may bind to RNA i.e. the test compound may be a RNA-binding compound. Suitable RNA-binding compounds may include RNA splicing modifiers, such as risdiplam and analogues thereof.

In other embodiments, the test compound may bind to a nucleic acid associated protein, for example a DNA associated protein or an RNA associated protein. The location within the nucleic acid of a nucleic acid associated protein that is bound by the test compound may be determined by a method described herein. Nucleic acid associated proteins may include histones; transcription factors, such as Sox2 and c-Myc; nucleases, such as DNAse and RNAse; polymerases; helicases; gyrases; DNA damage repair enzymes, such as PARP, ATM and Rad51; epigenetic modulators, such as histone deacetylases (HDACs), histone acetyltransferases, histone acetyltransferases, histone demethylases, histone methyltransferases, EZH2, DOT1L, protein arginine deiminases, and epigenetic reader domains, including bromodomains (BRDs), such as BRD2 and BRD4; DNA methyltransferases; kinases, such as CDK4, CDK6, CDK7, CDK9, AMP-activated protein kinase (AMPK), Aurora Kinase; Janus Kinase (JAKs) and protein kinase C; nuclear receptors, such as retinoic acid receptor, thyroid hormone receptor, progesterone receptor, glucocorticoid receptor, androgen receptor and estrogen receptor; transcriptional cofactors, epigenetic transcriptional cofactors and sirtuins.

In some preferred embodiments, the test compound may bind to a histone. Histones include histones H2A, H2B, H3, H4 (so-called core histones), H1/H5 (so called linker histone). The core histones associate to form an octamer that associates with nucleosomal DNA to form a nucleosome with the linker histone H1 binds the nucleosome at the entry and exit sites of the DNA. A histone-binding compound may bind to an unmodified histone or a histone modified by a histone mark, such as a methylated, which may be mono-, di- or tri-methylated, glycosylated, phosphorylated, ADP-ribosylated, acetylated, ubiquitinylated, SUMOylated or citrullinated histone (Luger, K. et al (1997) Nature 389, 251-260; (Ausio J (2001) Biochem Cell Bio 79, 693).

The test compound may bind covalently or non-covalently to the nucleic acid or nucleic acid associated protein.

In some preferred embodiments, the test compound binds non-covalently. For example, the test compound may non-covalently bind to chromatin with Kd of 0.001 nM or higher, 0.01 nM or higher, 0.1 nM or higher, 1 nM or higher, 5 nM or higher, 10 nM or higher, 15 nM or higher or 20 nM or higher. In some embodiments, the test compound may bind with an affinity of 0.1 nM to 20 μM, for example 1 nM to 10 μM or 5 nM to 1 μM.

In the methods described herein, the test compound may bind non-covalently to nucleic acid or nucleic acid associated protein without subsequent covalent cross-linking to the nucleic acid or nucleic acid associated protein.

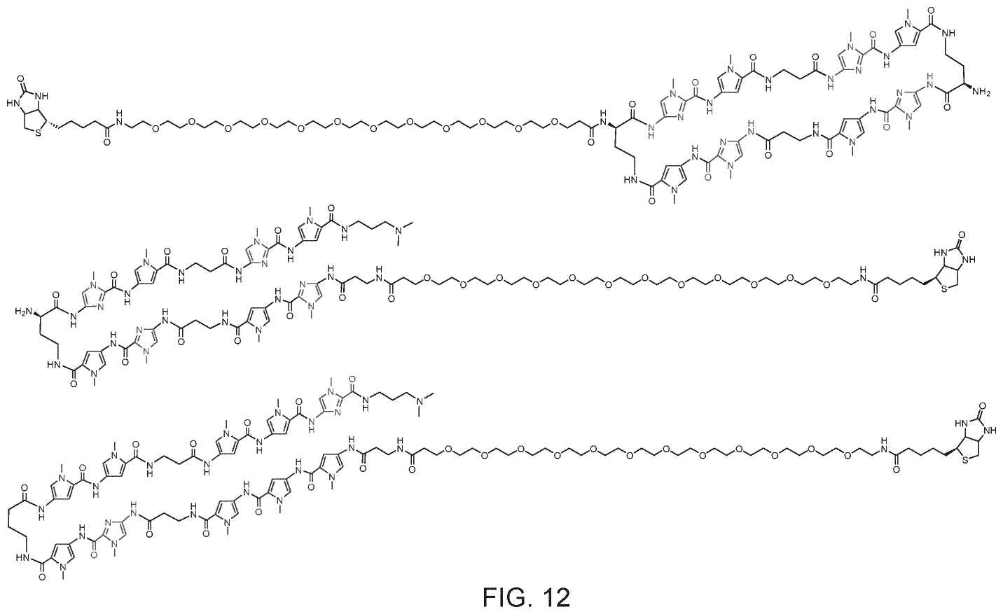

Suitable test compounds may include peptides, for example peptides of 50 amino acids or less, and small organic molecules of less than 5 KDa or less than 1 kDa, for example drugs, such as anti-cancer drugs. For example, the test compounds may include nuclear receptor inhibitors, such as estradiol, tamoxifene, raloxifene, dihydrotestosterone, bicalutamide, dexamethasone, retinoic acid, triiodothyronine, progesterone, mifepristone, and rosiglitazone; pyrrole-imidazole polyamides, such as PIP1, PIP2 and PIP2 (FIG. 12), kinase inhibitors, such as PD0332991, LEE011, THZ, AT7519, flavopiridol, and genistein; BET bromodomain inhibitors, such as JQ1 and iBET151; CDK9 inhibitors, such as AT7519; mechlorethamine, doxorubicin, actinomycin, bleomycin, etoposide, thalidomide, carboplatin, oxaliplatin mitomycin C, intercalating agents, such as cisplatin, bleomycin and adriamycin; G-quadruplex binding compounds, such as PDS and PhenDC; HDAC inhibitors, such as FK228, SAHA, LBH589 and valproic acid; Dot1L inhibitors such as EPZ004777; PARP inhibitors, such as olaparib, iniparib, rucaparib and velparib; and DNA polymerase inhibitors, such as amphidicolin.

The test compound may bind to the nucleic acid or a nucleic acid associated protein at one or more locations in the nucleic acid. The test compound may bind directly to the nucleic acid at one or more locations within the sequence of the nucleic acid; or the test compound may bind to nucleic acid associated proteins at one or more locations within the sequence of the nucleic acid. For example, nucleic acid associated proteins that contain binding sites for the test compound may be associated with the nucleic acid at one or more locations within the sequence of the nucleic acid.

The test compound is covalently linked to a tag to form a tagged test compound. The tag may be any label, molecule or group which allows the specific binding of a binding member to the test compound to which it is attached. The tag may allow covalent or non-covalent binding of the binding member. Suitable tags include immunogens, such as digoxigenin; short peptides, such as glutathione and FLAG™; or small organic compounds such as biotin and trimethoprim (TMP).

In some embodiments, a suitable tag may allow covalent binding of a binding member. Suitable tags may include click-based tags that react with a binding member through a click chemistry reaction. For example, a click-based tag may comprise a first click chemistry group. Suitable click chemistry groups are well-known in the art and may include one of an azide group or an alkyne group. The tag may be reacted with a first binding member comprising a second click chemistry group that reacts with first click chemistry group, for example the other of an azide group or an alkyne group, to covalently link the binding member to the tag.

Other suitable tags may include a HaloTag™ ligand, SNAP™ ligand, or CLIP™ ligand and may be covalently reactive with a first binding member that is a HaloTag™, SNAP™ tag, or CLIP™ tag, respectively.

Preferably, the tag is biotin and the tagged test compound may be a biotinylated test compound.

The tag may be connected directly to the test compound or may be connected to the test compound via a linker. Suitable linkers may include alkyl chains, PEG chains and combinations thereof. Suitable linkers may also include cleavable linkers, such as disulfides, Suitable linkers and methods of covalently linking the test compound and the tag are well-known in the art.

The first binding member (also referred to as the primary binding member) specifically binds to the tag moiety of the tagged test compound. For example, when the tag is biotin; the first binding member may specifically bind to biotin. Suitable first binding members for binding to a biotin tag may include anti-biotin antibodies, avidin and streptavidin.

A binding member is a molecule that binds specifically to a target molecule or ligand, such as a tag. The binding member may bind covalently or more preferably non-covalently to the tag of the tagged test compound. A binding member that specifically binds to a tag may not show any significant binding to molecules other than the tag. In particular, the binding member may show no significant binding to proteins, DNA or other antigens that may be present in a cell or cell extract. Generally, an antibody or other binding member which specifically binds to a tag may have a binding affinity (Ka) greater than about 10⁵ moles/litre (e.g. 10⁶ or more, 10⁷ or more, 10⁸ or more, 10⁹ or more, 10¹⁰ or more, 10¹¹ or more, or 10¹² or more moles/litre).

Suitable binding members may include antibodies, aptamers, enzymes, peptides or other binding modalities. In some preferred embodiments, a binding member may be an antibody, for example a whole antibody (e.g. IgG, such as IgG4) or an antibody fragment (e.g. single chain variable fragment (scFv), Fab, dAb, nanobody, single chain antibody, F(ab)₂ fragment, a VHH fragment, a VNAR fragment, or single-chain Fab fragment (scFab).

An antibody suitable for use as a first or second binding member (also referred to as primary and secondary binding members, respectively) in the present methods may be monoclonal or polyclonal and may be produced using conventional techniques. For example, an antibody may be produced by immunising a mammal (e.g. mouse, rat, rabbit, horse, goat, sheep or monkey) with the tag. Antibodies may be obtained from the immunised animals using any of a variety of techniques known in the art, and screened, preferably using binding of antibody to the tag. For instance, Western blotting techniques or immunoprecipitation may be used (Armitage et al. (1992) Nature 357:80-82). Isolation of antibodies and/or antibody-producing cells from an animal may be accompanied by a step of sacrificing the animal.

Monoclonal antibodies may be produced by isolating antibody producing cells from the immunised mammal, fusing them with immortalised cells to produce a population of antibody producing hybridoma cells. The population may then be screened to identify a hybridoma cell that produces an antibody which displays optimal binding characteristics. Methods of producing hybridoma cells and monoclonal antibodies are well known in the art (see, for example, Harlow et al Antibodies: A Laboratory Manual, Cold Spring Harbor Laboratory (Cold Spring Harbor, NY, 1988) pp. 353-355). As an alternative or supplement to immunising a mammal with a peptide, an antibody specific for a tag may be obtained from a recombinantly produced library of expressed immunoglobulin variable domains, e.g. using lambda bacteriophage or filamentous bacteriophage which display functional immunoglobulin binding domains on their surfaces; for instance see WO92/01047. The library may be naive, that is constructed from sequences obtained from an organism which has not been immunised with a peptide comprising the epitope or may be one constructed using sequences obtained from an organism which has been exposed to the antigen of interest.

Suitable binding members for use as a first or second binding members in the present methods are well-known in the art and may be produced using standard techniques or obtained from commercial sources.

The first binding member and tagged test compound may be contacted with the nucleic acid sequentially or at the same time.

In some embodiments, the tagged test compound may be contacted with nucleic acid, such that binding of the tagged test compound occurs at one or more locations within the nucleic acid sequence. This may be useful for example when the nucleic acid is present in a live cell, as described above. The first binding member may then be contacted with the nucleic acid, such that it binds to tagged test compound that is bound at the one or more locations.

In other embodiments, the tagged test compound and the first binding member may be contacted with nucleic acid at the same time. For example, a complex comprising the tagged test compound and the first binding member may be contacted with the nucleic acid. A method described herein may further comprise contacting the tagged test compound with the first binding member to form a complex before contacting the complex with chromatin. A method of mapping the locations of one or more binding sites of a test compound within a nucleic acid may comprise;
contacting the nucleic acid with a complex comprising a tagged test compound and a first binding member, wherein the tagged test compound comprises a test compound covalently linked to a tag and binds to the nucleic acid or to protein associated with the nucleic acid at one or more locations within the nucleic acid; and the first binding member is specifically bound to the tag,
contacting the nucleic acid with a second binding member that specifically binds to the first binding member and is attached to an activatable nuclease, such that the second binding member binds to first binding member that is bound to the tagged test compound at the one or more binding sites,
activating the nuclease, such that the nuclease cleaves the nucleic acid at the one or more binding sites to generate fragments, and;
determining the sequences of the generated fragments.

Washing steps may be performed as required to remove unbound reagents. For example, test compound that is not bound to chromatin after the contacting step may be removed by washing. Similarly, first binding member that is not bound to chromatin via the test compound after the contacting step may also be removed by washing. Suitable methods of washing are well known in the art. For example, washing may be performed using a buffer, such as 20 mM HEPES pH7.5, 150 mM NaCl and 0.5 mM spermidine, as described herein.

The second binding member (also referred to as a secondary binding member) binds specifically to the first binding member. For example, the second binding member may be an antibody. Suitable anti-immunoglobulin (Ig) antibodies are well known in the art. For example, the first antibody may be from a first species, for example a mammalian species, such as rabbit, and the second antibody may bind to antibodies from the first species. For example, the second antibody may be from a second species, for example a different mammalian species, such as guinea pig.

The second binding member may be covalently or non-covalently attached to the nuclease. In some embodiments, the second binding member may be non-covalently bound to the transposase. Suitable methods for non-covalent binding of a nuclease to a binding member are well-known in the art. For example, the second binding member may be an antibody and the nuclease may be fused to an immunoglobulin binding moiety in a fusion protein. The fusion protein may be non-covalently bound to the second antibody through the immunoglobulin binding moiety. Suitable Ig binding moieties are well-known in the art and may include protein A, protein G protein L, protein Y, or binding domains of any of these, an anti-Ig antibodies or antibody molecules, such as single chain antibodies, Fab fragments, F(ab)₂ fragments, a VHH fragments, a VNAR fragments, nanobodies, single chain variable fragments (scFvs), or single-chain Fab fragments (scFabs). In other embodiments, the second binding member may be covalently bound to the nuclease. For example, the second binding member and the transposase may be contained in a single fusion protein.

A nuclease is an enzyme capable of cleaving the phosphodiester bonds between nucleotides of nucleic acids. Suitable nucleases may include transposases. The nuclease may be an activatable nuclease. An activable nuclease may be converted from an inactive state to an active state by modification of the conditions. For example, a magnesium-activated transposase may be converted into an active state by increasing the concentration of magnesium ions (Mg²⁺) or manganese ions (Mn²⁺), for example to a concentration within range of about 0.1 mM to about 10 mM. A calcium-activated micrococcal nuclease may be converted into an active state by increasing the concentration of calcium ions (Ca²⁺). Suitable activatable nucleases are available in the art (see for example U.S. Pat. No. 9,005,935B2 and WO2022056309A1).

Suitable activatable nucleases are well known in the art and include micrococcal nuclease (MNase) and transposases, such as Tn5, Tn7, Mu, IS5 and IS91 (for example U.S. Pat. No. 9,005,935B2, Mizuuchi, K., Cell, 35:785, 1983; Savilahti, H, et al, EMBO J., 14:4893, 1995, Goryshin and Reznikoff, J. Biol. Chem, 273:7367 (1998) and WO2022056309A1). In some preferred embodiments, the nuclease may be a transposase, such as a Tn5 transposase. A transposase may be loaded with one or more oligonucleotide adapters. For example, oligonucleotide adapters may be non-covalently bound to the transposase to form a complex comprising the transposase and the one or more oligonucleotide adapters. Oligonucleotide adapters are described below. Suitable methods for loading transposase with oligonucleotide adapters are well-known in the art. For example, the oligonucleotide adapters may be incubated with the transposase at room temperature for 1 hour. In some embodiments, the sequences of the labelled fragments may be determined by sequencing and the oligonucleotide adapters may be sequencing adapters. A transposase may be loaded with one or more sequencing adapters. For example, sequencing adapters may be non-covalently bound to the transposase to form a complex comprising the transposase and the one or more sequencing adapters. Sequencing adapters are described below. Suitable methods for loading transposase with sequencing adapters are well-known in the art. For example, the sequencing adapters may be incubated with the transposase at room temperature for 1 hour.

Second binding member that is not bound to the first binding member after the contacting step may be removed by washing.

Following the binding of the second binding member to the first binding member, the nuclease may be activated. Suitable methods of activating nuclease are well-known in the art. For example, a nuclease may be activated by increasing the concentration of the appropriate ion, for example magnesium or manganese ions for transposase and calcium for micrococcal nuclease. The activated nuclease cleaves the nucleic acid at locations within the nucleic acid at which the tagged test compound is bound to the nucleic acid or to a nucleic acid associated protein to generate nucleic acid fragments. For example, the nuclease may cleave the nucleic acid within 10 bp to 1000 bp of the location of the binding site, preferably 50 bp to 500 bp. The nucleic acid fragments include the sequence of the nucleic acid at the locations where the tagged test compound is bound to the nucleic acid or to a nucleic acid associated protein.

Specific cleavage of the nucleic acid by the nuclease at locations where the tagged test compound is bound generates a specific population of fragments that contain the nucleic acid sequence at these locations. This allows the nucleic acid sequence at these locations to be determined by subsequent analysis. The sequence at these locations may be indistinguishable from other sequence when the nucleic acid is randomly fragmented (low signal to noise).

In some embodiments, oligonucleotide adaptors, such as sequencing adaptors, may be attached to the ends of the nucleic acid fragments, for example by ligation, to generate nucleic acid fragments labelled at each end with an oligonucleotide adaptor. Suitable ligation methods are well-known in the art.

Oligonucleotide adaptors may be double stranded oligonucleotides, partially double stranded oligonucleotides or single stranded oligonucleotides that are attached to nucleic acid molecules to allow the sequence of the nucleic acid molecule to be determined. Oligonucleotide adaptors may include amplification adaptors, and sequencing adaptors. Amplification adaptors are double stranded, partially double stranded or single-stranded oligonucleotides that are attached to nucleic acid molecules to allow the molecules to be amplified, for example by PCR. Suitable amplification adaptors may specifically hybridise to amplification primers and are readily available in the art. Sequencing adaptors are double stranded, partially double stranded or single stranded oligonucleotides that are attached to nucleic acid molecules to allow sequencing. Suitable sequence adaptors for any method of sequencing are available in the art. In some embodiments, a sequencing adaptor may comprise a primer recognition sequence. A primer recognition sequence is a nucleotide sequence that is complementary to the sequence of an amplification primer. The presence of primer recognition sequences allows the labelled fragments to be amplified, for example by PCR using amplification primers that target the primer recognition sequences. The primer recognition sequences may be heterologous sequences that are not naturally present in the chromatin DNA sequence. Suitable primer recognition sequences are well-known in the art and include Illumina-compatible barcoded i7/i5 primers.

In some embodiments, the sequencing adaptors may comprise a first and a second sequencing adaptor. The first sequencing adaptor may comprise a first primer recognition sequence. The second sequencing adaptor may comprise a second primer recognition sequence.

In some embodiments, the sequencing adaptors may further comprise a barcode. The barcode is a nucleotide sequence that is unique for the sample from which the population of nucleic acids is obtained. A suitable barcode sequence may be 6-10 nucleotides. The barcode allows sequence reads from a specific sample to be unambiguously identified in a pooled multiplex sequencing reaction. Each sample may have a unique barcode, so that all of the nucleic acids from the same sample receive the same barcode. Once prepared, populations of nucleic acids from different samples may be mixed into a single pool and sequenced. The sample from which a sequence read from the pool originates may then be identified from the barcode. For example, a suitable barcode for the multiplex sequencing of 24 samples (a 24-plex reaction) may consist of at least 6 nucleotides, preferably 6 nucleotides (Craig D W et al. 2008. Nat Methods 5, 887; Cronn R et al. 2008. Nucleic Acids Res, 36, e122). The use of barcodes in sequencing reactions is well-known in the art. For example, the Illumina sequencing 8-mer barcodes i5 and i7 may be used. In embodiments in which the nuclease is an endonuclease, such as MNase, oligonucleotide adaptors, such as sequencing adaptors, may be attached to the ends of the nucleic acid fragments, for example by ligation.

In embodiments in which the nuclease is a transposase, the transposase may be loaded with oligonucleotide adaptors, such as sequencing adaptors. The transposase may cleave the nucleic acid and insert the oligonucleotide adaptors at the ends of the nucleic acid fragments. This generates nucleic acid fragments labelled at each end with an oligonucleotide adaptor and may be referred to herein as “tagmentation”. Oligonucleotide adaptors suitable for attachment to a transposase may be double stranded oligonucleotides that comprise a transposase recognition sequence and a primer recognition sequence. A transposase recognition sequence is a sequence that is targeted for transposition by a transposase. For example, inverted transposase recognition sequences may bracket the transposon sequence that is inserted by a transposase. The sequence of the transposase recognition sequence may depend on the transposase. Suitable transposase recognition sequences for transposases are well known in the art. For example, suitable transposase recognition sequences for Tn5 may include 19-mer end sequences, such as outside end (OE) sequences, such as 5′-CTG ACT CTT ATA CAC AAG T-3′ and inside end (IE) sequences, such as 5′-CTG TCT CTT GAT CAG ATC T-3′; and mosaic end (ME) sequences, such as 5′-CTG TCT CTT ATA CAC ATC T-3′. In some preferred embodiments, 19-mer ME sequences may be employed. In some embodiments, the oligonucleotide adaptors may comprise a first and a second oligonucleotide adaptor. The first oligonucleotide adaptor may comprise a first transposase recognition site and a first primer recognition sequence. The second oligonucleotide adaptor may comprise a second transposase recognition site and a second primer recognition sequence. For example, the sequencing adaptors may comprise a first and a second sequencing adaptor. The first sequencing adaptor may comprise a first transposase recognition site and a first primer recognition sequence. The second sequencing adaptor may comprise a second transposase recognition site and a second primer recognition sequence.

In some embodiments, labelled nucleic acid fragments produced as described above may be amplified to directly determine the sequence of the fragments. The labelled nucleic acid fragments may be amplified using one or more primers that hybridise to a nucleic acid sequence that is known to comprise binding sites for the test compound. The amplification of a labelled nucleic acid fragment with the primers to generate an amplified fragment is indicative that the labelled nucleic acid fragment comprises the nucleic acid sequence. For example, the labelled nucleic acid fragments may include nucleotide sequences to which the compound directly binds within the nucleic acid; or the nucleotide sequences that are associated with (or contained within the same or an adjacent nucleosome as) nucleic acid associated proteins to which the compound binds.

Suitable nucleic acid sequences include sequences of RNA, for example nuclear RNA, such as pre-mRNA and miRNA, and genomic DNA, such as chromosomal or mitochondrial DNA. Nucleic acid sequences may include binding sites, genes and transcripts associated with biological responses to the test compound and/or off-target toxicity. Suitable binding sites, genes and transcripts may for example be known in the art or may be previously determined by the methods described herein. Preferably, the labelled nucleic acid fragments are amplified using multiple sets of primers that each hybridise to a different nucleic acid sequence that is known to comprise binding sites for the test compound. This allows the presence of multiple nucleic sequences within the labelled nucleic acid fragments to be determined. The multiple nucleic acid sequences hybridised by the sets of primers may form a panel. For example, a panel targeted by the sets of primers may comprise 2,3 4, 5, 6, 7,8, 9, 10 or more, 100 or more or 1000 or more nucleic acid sequences. The panel may be useful for example as a “fingerprint” or “signature” to allow the rapid identification of test compounds with desirable characteristics and reduced off-target toxicity. Preferably, labelled nucleic acid fragments are amplified quantitively, such that the amount or number of copies of a nucleic acid sequence in the labelled nucleic acid fragments may be determined. This may for example allow the amount of binding of the test compound to each nucleic acid sequence to be determined. Suitable amplification methods are well established in the art and include quantitative polymerase chain reaction (qPCR), reverse transcription PCR (RT-PCR), and isothermal amplification method.

A sample binding profile may be generated from the distribution of the nucleic acid sequences amplified by the multiple sets of primers. The sample binding profile may comprise a set of scores or values indicative of the number or density of each amplified nucleic acid sequence. The number or density of nucleic acid sequences comprising a binding site may be indicative of the amount or extent of binding of the test compound at that site or the occupancy of that site by the test compound. The sample binding profile may therefore reflect test compound binding, occupancy, or distribution at a range of binding sites defined by the panel of nucleic acid sequences that are amplified by the multiple sets of primers. The sample binding profile may be expressed in any convenient format, for example numerically or graphically.

In other embodiments, labelled nucleic acid fragments produced as described above may be amplified to produce amplified fragments for sequencing. The labelled nucleic acid fragments may be amplified using primers that hybridise to the primer recognition sequences of the sequencing adaptors. Suitable amplification methods are well established in the art and include polymerase chain reaction (PCR) (reviewed for instance in “PCR protocols; A Guide to Methods and Applications”, Eds. Innis et al, 1990, Academic Press, New York, Mullis et al, Cold Spring Harbor Symp. Quant. Biol., 51:263, (1987), Ehrlich (ed), PCR technology, Stockton Press, NY, 1989, and Ehrlich et al, Science, 252:1643-1650, (1991)).

In some embodiments, the nucleic acid fragments or amplified nucleic acid fragments may be extracted, for example from the sample. Suitable methods for extracting and isolating nucleic acid from samples of biological fluid are well-known in the art and include phenol/chloroform extraction and alcohol precipitation, caesium chloride density gradient centrifugation, solid-phase anion-exchange chromatography and silica membrane-based techniques (e.g. Quick-cfDNA™, Zymo Research Corp). Many known techniques and protocols for nucleic acid extraction, amplification, and sequencing as described herein, are known in the art, for example, Molecular Cloning: a Laboratory Manual: 3rd edition, Russell et al., 2001, Cold Spring Harbor Laboratory Press; Protocols in Molecular Biology, Second Edition, Ausubel et al. eds. John Wiley & Sons, 1992); Next-generation Sequencing: Current Technologies and Applications; ed Jianping Xu Caister Academic Press (2014)

Amplification may generate a population or library of amplified fragments for sequencing. Each amplified fragment in the population or library may contain the nucleic acid sequence at the location of a binding site of the test compound. A population or library of amplified fragments generated as described herein may be purified before sequencing using standard techniques, including spin-column chromatography (e.g. Ampure XP™ beads)

The labelled fragments or amplified fragments may be sequenced using standard sequencing techniques. Suitable techniques include using any convenient low or high throughput sequencing technique or platform, including Sanger sequencing, Solexa-Illumina sequencing, ligation-based sequencing (SOLID™), pyrosequencing; single molecule real-time sequencing (SMRT™); PacBioscience sequencing; and semiconductor array sequencing (Ion Torrent™). Preferably, sequencing is performed by a next-generation sequencing technique. Suitable protocols, reagents and apparatus for nucleic acid sequencing are well-known in the art and are available commercially.

The sequences of the nucleic acid fragments may be indicative of the sequence of the nucleic acid at the locations of the binding sites of the test compound. For example, the sequences of the amplified fragments may include the nucleotide sequences to which the compound directly binds within the nucleic acid; or the nucleotide sequences that are associated with (or contained within the same or an adjacent nucleosome as) nucleic acid associated proteins to which the compound binds. The position of the one or more binding sites within the nucleic acid may be identified or mapped. For example, the position of one or more binding sites within the genome of a cell, such as a eukaryotic, mammalian or human cell, may be identified or mapped by a method described herein.

In some embodiments, a set of sequence reads of nucleic acid fragments may be generated by sequencing, for example 1000 or more, 10,000 or more, 100,000 or more, 1000,000, 10,000,000 or more, or 100,000,000 or more, or 1000, 000,000 or more sequence reads may be generated. The sequence reads may be analysed by routine bioinformatic techniques. Suitable techniques are well-known in the art. For example, duplicate reads, low quality sequence reads and reads arising only from sequencing adaptors may be removed.

In some embodiments, the nucleic acid fragment sequence reads in the population may be analysed for the presence of binding sites and/or patterns of binding sites. The nucleic acid fragment sequence reads in the population may further be analysed for the presence of other features, such as mutations, epigenetic modifications or sequence motifs, that are associated with the binding sites and/or patterns of binding sites.

The nucleic acid fragment sequence reads in the population may be mapped to one or more locations in a reference genome. Suitable reference genomes are available in the art. For example, human nucleic acid fragment sequence reads in the population may be mapped to locations in the sequence of the human genome. In some embodiments, the reference genome may be matched to the gender, ethnicity and/or other characteristics of the individual from whom the sample is obtained.

The nucleic acid fragment sequence reads in the population may be mapped by aligning sequence reads in the population with the sequence of the reference genome, for example the human genome. The location of the sequence reads within the reference genome may be identified. Suitable software tools for mapping populations of sequence reads within the genome are readily available in the art.

The distribution of the nucleic acid fragment sequence reads in the population within the genome or at set of sites or loci within the genome (i.e. the number of sequence reads that map to each location within the genome or set of sites or loci) may be determined from the locations of the sequence reads in the population. Optionally, the distribution of the amplified fragment sequence reads may be subjected to mathematical transformation. Suitable transformations include Fourier transformation.

A sample binding profile may be generated from the distribution or transformed distribution of the nucleic acid fragment sequence reads. The sample binding profile may comprise a set of scores or values indicative of the number or density of nucleic acid fragment sequence reads that map to each location or position within the genome (i.e. a genome wide plot) or set of sites or loci within the genome. The number or density of nucleic acid fragment sequence reads that map to a location or position in genome may be indicative of the amount or extent of binding of the test compound at that location or position or the occupancy of that location or position by the test compound. The sample binding profile may therefore reflect test compound binding, occupancy, or distribution in the genome or target loci within the genome. The sample binding profile may be expressed in any convenient format, for example numerically or graphically.

In some embodiments, the sample binding profile may be used to identify the locations of binding sites that are associated with biological responses to the test compound and may be useful in determining or predicting the response of an individual to treatment with the test compound. The sample binding profile may be used for therapeutic stratification, the optimization of combination therapies, diagnosis of a disease condition, or the determination of side-effects of treatment with a test compound.

A marker location is a position in the genome at which the binding of the test compound varies between different sources i.e. binding at the marker location by the test compound is source specific, for example from different individuals, tissues or cell-types. Binding of the compound at a set of marker locations in a sample binding profile may provide a signature that is characteristic of the source.

In some embodiments, suitable marker locations may be identified by determining binding at a plurality of candidate locations in reference binding profiles from a set of reference sources. The reference binding profiles may comprise a set of scores or values indicative of binding at the set of marker locations in genomic DNA from a known source, for example, a specific known tissue or cell type. The reference binding profile may reflect the binding at the locations in genomic DNA from the known source. Suitable reference binding profiles may be obtained or generated by routine experimentation using known tissues or cell type or produced from publicly available data sources, such as databases of genomic information.

A candidate location may be identified as a marker binding location if the binding occupancy at the site is higher or lower for one reference source in the set than for the other sources in the set. For example, binding at the location may be higher or lower than the mean binding at the location in the other reference sources in the set. In some embodiments, binding at the location in one reference source may be above or below a predetermined threshold value relative to the mean binding at the location in the other reference sources in the set.

In other embodiments, suitable marker locations may be identified by providing a first set of sample binding profiles from control individuals, for example healthy individuals, and a second set of sample binding profiles from test individuals, for example individuals with a disease condition, such as a specific cancer, or individuals known to be responsive or non-responsive to the test compound. The binding or occupancy of the compound at a plurality of candidate locations in the first and second sets of sample binding profiles may be compared. A candidate location may be identified as a marker location if the binding or occupancy of the compound at the location is higher or lower in the first set of sample binding profiles than the second set of sample binding profiles. For example, mean binding or occupancy of the compound at the location may be higher or lower in the first set than the second set. In some embodiments, mean binding or occupancy of the test compound at the location in the first set may be above or below a predetermined threshold value relative to the mean binding or occupancy of the compound at the location in the second set.

A candidate location may also be identified as a marker location if it is found to be in linkage disequilibrium with a site at which binding or occupancy of the test compound at is higher or lower for one reference source in the set than for the other sources in the set.

A sample binding profile from an individual may be used to determine binding or occupancy of the test compound at a set of marker locations in the individual. This may be useful in determining the effect of the test compound on the individual or the responsiveness of the individual to the test compound, for example, to determine the effectiveness of treatment with the test compound in the individual or to determine the suitability of the individual for treatment with the test compound. A sample binding profile from an individual may also be used to demonstrate the mechanism of effect of the test compound; as part of a clinical trial; or as evidence for off-target toxicity, where the toxicity is caused by binding to additional/alternative locations of binding within the nucleic acid.

In some embodiments, the methods described above may be used in multiplex assays to map the locations of the binding sites of more than one test compound. For example, a method of mapping the locations of binding sites of a population of test compounds within a nucleic acid may comprise;
(i) contacting the nucleic acid with a population of tagged test compounds, each tagged test compound in the population comprising a test compound covalently linked to a tag, wherein the tagged test compounds in the population bind to the nucleic acid or to protein associated with the nucleic acid at one or more sites within the nucleic acid,
(ii) contacting the tagged test compounds with a population of first binding members, such that each first binding member in the population specifically binds to a different tagged test compound,
(iii) contacting the nucleic acid with a population of second binding members attached to activatable nucleases, such that each second binding member in the population specifically binds to a different first binding member that is bound to a tagged test compound at a binding site in the nucleic acid,
(iv) activating the nucleases, such that the nucleases cleave the nucleic acid at the one or more binding sites to generate fragments, and;
(v) determining the sequences of the generated fragments.

The sequences of nucleic acid fragments generated by a nuclease bound via the second and first binding members to a tagged test compound in the population may be indicative of the locations of the one or more binding sites within the nucleic acid of the test compound.

A population may comprise two or more, three or more, four or more, five or more, six or more, seven or more, eight or more, nine or more or ten or more members.

In some embodiments, each tagged test compound in the population may be contacted individually with a first binding member to form a complex, and the complexes combined to produce a population of complexes comprising a first binding member and a tagged test compound. A different first binding member may be attached to each tagged test compound in the population. The population of complexes may then be contacted with the nucleic acid.

In other embodiments, each tagged test compound in the population may have a different tag. The population of first binding members may comprise first binding members that bind specifically to the different tags of the tagged test compound in the population, such that a different first binding member is attached to each tagged test compound in the population.

The population of second binding members may comprise second binding members that bind specifically to each different first binding member in the population of first binding members, such that a different second binding member is attached via the first binding member to each tagged test compound in the population.

In some embodiments, a different nuclease may be attached to each different second binding member, such that fragments generated by each nuclease can be distinguished.

In other embodiments, different oligonucleotide adaptors may be loaded onto the nuclease attached to each different second binding member, such that fragments generated by each nuclease are labelled with a different adaptor and can be distinguished. For example, the nuclease may be a transposase, and the transposase attached to each different second binding member may be loaded with a different oligonucleotide adaptors. The transposase attached via the first and second binding members to the tagged test compound may cleave the nucleic acid at a binding site of the tagged test compound and insert the oligonucleotide adaptors at the ends of the nucleic acid fragments. This generates nucleic acid fragments labelled at each end with an oligonucleotide adaptor. Because each different second binding member is loaded with a different oligonucleotide adaptor, the sequence of the oligonucleotide adaptor that is attached to each nucleic acid fragment can be used to identify the tagged test compound whose binding site is indicated by the sequence of the nucleic acid fragment.

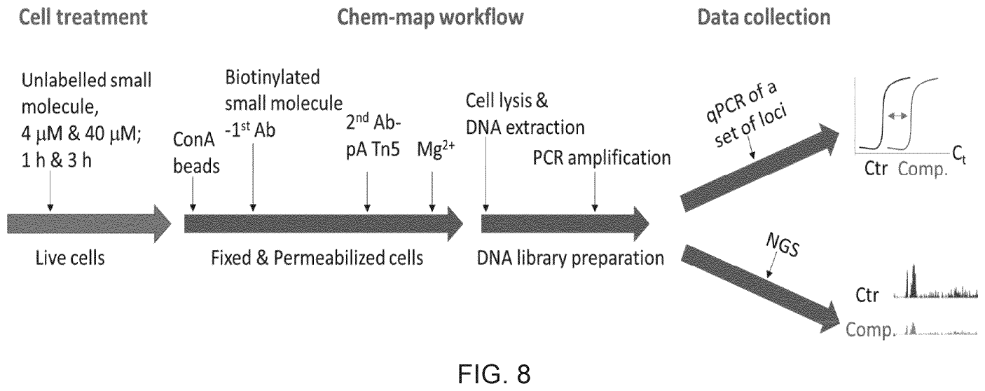

In some embodiments, the methods described above may be used in competitive assays. An example of a competitive assay as described herein is shown in FIG. 8. Suitable assays may comprise contacting a nucleic acid with a tagged test compound and an untagged second test compound. For example, a method of mapping the locations of one or more binding sites of a test compound within a nucleic acid may comprise;
(i) contacting the nucleic acid with a tagged test compound comprising a test compound covalently linked to a tag and an untagged second test compound, wherein the tagged test compound and optionally the untagged second test compound bind to the nucleic acid or to protein associated with the nucleic acid at one or more locations within the nucleic acid,
(ii) contacting the nucleic acid with a first binding member that specifically binds to the tag, such that the first binding member binds to the tagged test compound,
(iii) contacting the nucleic acid with a second binding member that specifically binds to the first binding member and is attached to an activatable nuclease, such that the second binding member binds to first binding member that is bound to the tagged test compound at the one or more binding sites,
(iv) activating the nuclease, such that the nuclease cleaves the nucleic acid at the one or more binding sites to generate fragments, and;
(v) determining the sequence of the generated fragments.

The tagged test compound and the untagged second test compound may be contacted with the nucleic acid simultaneously or sequentially. For example, the nucleic acid may be contacted with the untagged second test compound followed by the tagged test compound; the tagged test compound followed by the untagged second test compound; or both the untagged second test compound and the tagged test compound at the same time.

The test compound and the second test compound may be same or different (i.e. they may be the same chemical compound or different chemical compounds).

In some embodiments, the test compound and the second test compound may be peptides or small organic molecules as described above. In other embodiments, the test compound may be a peptide or small organic molecule as described above and the second test compound may be a protein, such as an antibody; a nucleic acid or other macromolecule. For example, the second test compound may be a polypeptide of greater than 50 amino acids or an organic molecule of greater than 5 KDa.

Also provided are kits for use the methods described herein and the use of such kits in such methods. A kit may comprise;
(i) a tag covalently linked or linkable to a test compound.
(ii) a first binding member that specifically binds to the tag
(iii) a second binding member that specifically binds to the first binding member,
(iv) a nuclease that is attached or attachable to the second binding member.

Suitable kit components are described above. A linkable tag is a tag can be covalently attached to a test compound. Suitable linkable tags include tags that comprises a first click chemistry group. Suitable click chemistry groups are well-known in the art and may include one of an azide group or an alkyne group. The linkable tag may be reacted with a test compound comprising a second click chemistry group that reacts with first click chemistry group, for example the other of an azide group or an alkyne group, to covalently link the compound to the tag.

The kit may further comprise (v) one or more sequencing adaptors. The sequencing adaptors may be attached or attachable to a transposase.

The kit may further comprise suitable buffers and washing solutions; fixing agents and permeabilising agents.

The kit may further comprise a solid support. Suitable solid supports may comprise a capture molecule that binds to eukaryotic cells, such as a lectin.

The kit may further comprise nucleic acid extraction and purification reagents. Suitable reagents are well-known in the art and include spin-chromatography columns.

The kit may further comprise amplification reagents. Suitable reagents are well-known in the art and include primers, dNTPs, and thermostable polymerases. In some embodiments, a kit may comprise amplification primers for amplification of one or more regions or loci of interest in a genome.

The kit may include instructions for use in a method described herein.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

### Experimental

In this application, we disclose a generalized approach to establishing interaction maps for small molecules that bind to the genomic DNA or to chromatin proteins in situ. We exemplify the approach with 3 distinct classes of DNA or protein interacting molecules. We use the bromodomain inhibitor JQ1 to validate our method in small samples of cells with high signal-to-noise ratio. We have then mapped genomic target sites in cells for 3 widely used chemicals for the first time, including 2 benchmarking molecules that bind G-quadruplex DNA and first-line anticancer drug Doxorubicin. Finally, we examined the influence of HDACi on Doxorubicin binding on genomic DNA.

### Materials and Methods

### General

Chemicals and reagents were purchased from Sigma-Aldrich, MedChemExpress, Fluorochem. All organic solvents were distilled by standard purification methods before use or purchased as anhydrous from Sigma-Aldrich. All reactions were performed in oven-dried glassware under argon unless otherwise stated. NMR spectra were recorded on a Bruker 400 MHz Advance III HD Spectrometer or a 500 MHz DCH Cryoprobe Spectrometer operating at 400 and 500 MHz for 1H NMR and 125 MHz 13C NMR respectively in DMSO-d₆.

NMR data are reported as follows: chemical shifts in parts per million (ppm) referring to the solvent residual peak, multiplicities (s=singlet, d=doublet, t=triplet, q=quartet; m=multiplet, br=broad) and coupling constant values in Hz. LC-MS was performed on an Amazon ESI-MS (Bruker) connected to a Dionex UltiMate 3000 UHPLC system (Thermo Fisher Scientific). High-resolution mass spectra (HRMS) were obtained from a Waters Vion IMS QTOF spectrometer. Flash column chromatography was performed using CombiFlash Rf (Teledyne ISCO) with C18 puriFlash columns (Interchim). Pyridostatin (PDS) and PhenDC3 were prepared according to the previously reported procedures.

### Chemical Synthesis

### 1. Synthesis of JQ1-bio

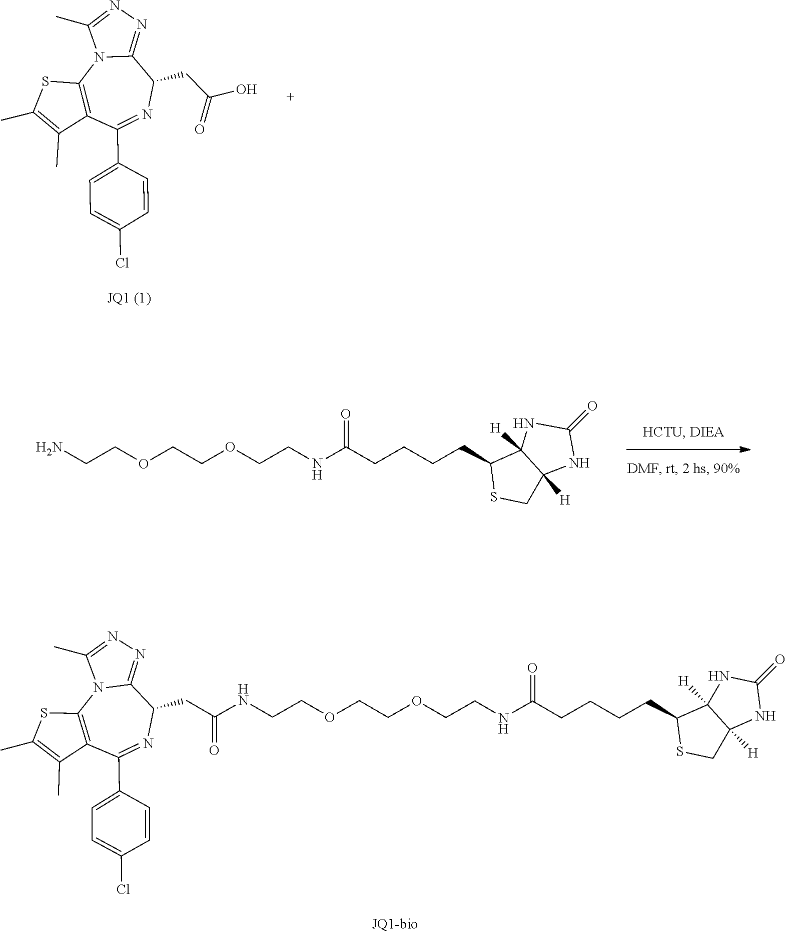 [SMILES: Cc1sc2c(c1C)C(c1ccc(Cl)cc1)=N[C@@H](CC(=O)O)c1nnc(C)n1-2 ; NCCOCCOCCNC(=O)CCCC[C@@H]1SC[C@H]2NC(=O)N[C@@H]12 ; Cc1sc2c(c1C)C(c1ccc(Cl)cc1)=N[C@@H](CC(=O)NCCOCCOCCNC(=O)CCCC[C@@H]1SC[C@@H]3NC(=O)N[C@@H]31)c1nnc(C)n1-2]

JQ1 (1, 10 mg, 25 μmol) and HCTU (12.4 mg, 30 μmol) were dissolved in 1 mL anhydrous DMF. Biotin-PEG2-NH2 (11.2 mg, 30 μmol) was added to the mixture and DIEA (23 μl, 125 μmol) was added, and the reaction was stirred for 2 hs in room temperature under argon. The solvent was evaporated to dryness in vacuo and the product was purified using flash column chromatography (C18 column, gradient elution: water (0.1% (v/v) TFA) to MeCN (0.1% (v/v) TFA) over 30 min at a flow rate of 18 mL/min). Solvents were removed through freeze-drying to obtain the product as a white powder (JQ1-bio, 17 mg, yield 90%). ¹H NMR (500 MHz, DMSO) δ8.27 (t, J=5.7 Hz, 1H), 7.82 (t, J=5.7 Hz, 1H), 7.48 (d, J=8.8 Hz, 2H), 7.44-7.39 (m, 2H), 6.40 (s, 1H), 4.51 (dd, J=8.0, 6.3 Hz, 1H), 4.31-4.25 (m, 1H), 4.10 (dd, J=7.7, 4.4 Hz, 1H), 3.51 (tt, J=5.3, 2.8 Hz, 4H), 3.44 (t, J=5.9 Hz, 2H), 3.39 (t, J=6.0 Hz, 2H), 3.33-3.12 (m, 7H), 3.07 (ddd, J=8.6, 6.2, 4.4 Hz, 1H), 2.79 (dd, J=12.4, 5.1 Hz, 1H), 2.59 (s, 3H), 2.55 (d, J=12.4 Hz, 1H), 2.42-2.38 (m, 3H), 2.04 (t, J=7.4 Hz, 2H), 1.61 (s, 3H), 1.59-1.53 (m, 1H), 1.51-1.40 (m, 3H), 1.27 (dtd, J=15.6, 8.8, 5.9 Hz, 2H). ¹³C NMR (126 MHZ, DMSO) δ172.59, 170.07, 163.61, 163.15, 155.50, 150.42, 137.08, 135.76, 132.65, 131.34, 130.66, 130.33, 130.06, 128.92, 70.03, 70.00, 69.65, 69.63, 61.48, 59.64, 55.86, 54.20, 40.13, 40.05, 39.96, 39.88, 39.79, 39.08, 38.89, 37.85, 35.54, 28.64, 28.48, 25.70, 14.51, 13.14, 11.74. HRMS (ESI-QTof) m/z: [M+H]⁺ calculated for C₃₅H₄₆ClN₈O₅S₂⁺: 757.3660; found, 757.2702.

### 2. Synthesis of PDS-bio

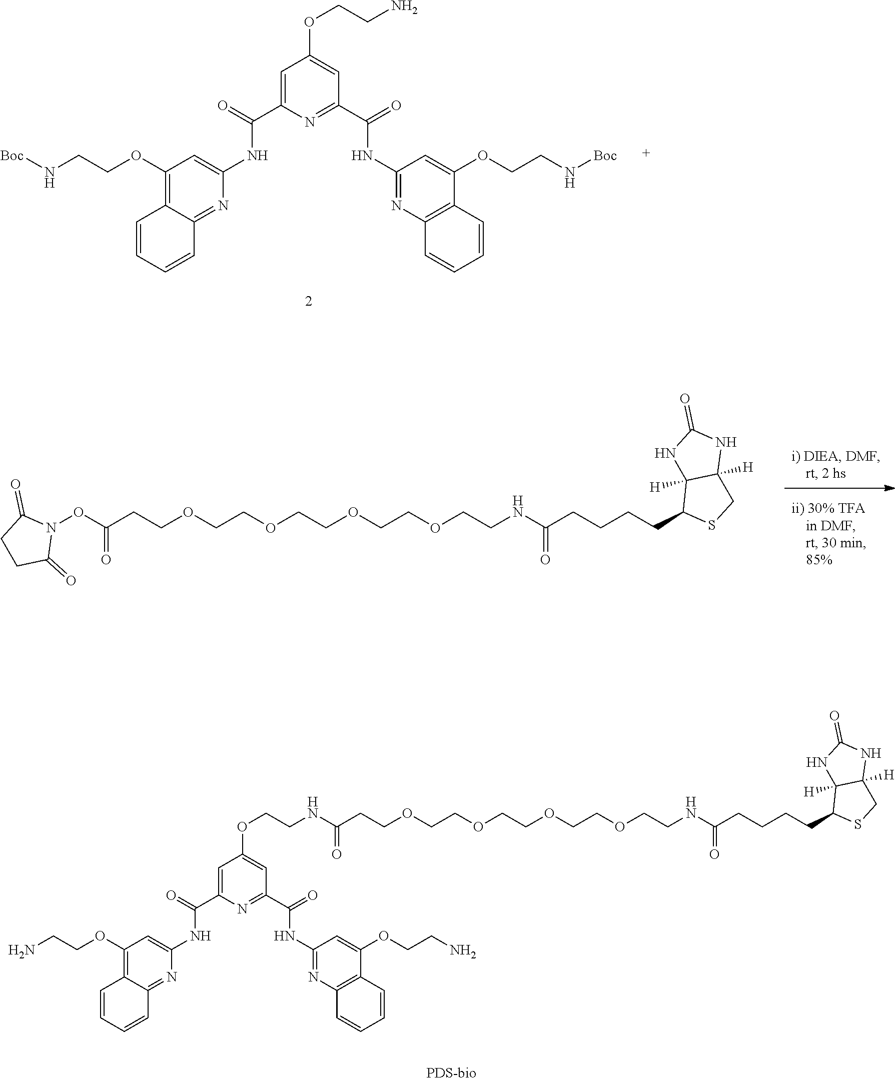 [SMILES: CC(C)(C)OC(=O)NCCOc1cc(NC(=O)c2cc(OCCN)cc(C(=O)Nc3cc(OCCNC(=O)OC(C)(C)C)c4ccccc4n3)n2)nc2ccccc12 ; O=C(CCCC[C@@H]1SC[C@@H]2NC(=O)N[C@@H]21)NCCOCCOCCOCCOCCC(=O)ON1C(=O)CCC1=O ; NCCOc1cc(NC(=O)c2cc(OCCNC(=O)CCOCCOCCOCCOCCNC(=O)CCCC[C@@H]3SC[C@@H]4NC(=O)N[C@@H]43)cc(C(=O)Nc3cc(OCCN)c4ccccc4n3)n2)nc2ccccc12]

The synthesis of di-boc protected PDS (2) has been reported previously (X. Zhang, et al. Nature Chem., 13 (2021) 626-633). Di-boc protected PDS (20 mg, 25 μmol) and Biotin-PEG4-OSu (17.6 mg, 30 μmol) were dissolved in 1 mL anhydrous DMF. DIEA (23 μl, 125 μmol) was added, and the reaction was stirred for 2 hs in room temperature under argon. The solvent was evaporated to dryness in vacuo. The crude was dissolved in 1 mL 30% TFA in DMF and stirred for 30 min in room temperature and the solvent was evaporated to dryness in vacuo. The product was purified using flash column chromatography (C18 column, gradient elution: water (0.1% (v/v) TFA) to MeCN (0.1% (v/v) TFA) over 30 min at a flow rate of 18 mL/min)). Solvents were removed through freeze-drying to obtain the product as a white powder (PDS-bio, 22.7 mg, 85%). ¹H NMR (500 MHz, DMSO) δ12.08 (s, 2H), 8.42 (dd, J=8.3, 1.5 Hz, 2H), 8.18 (t, J=5.6 Hz, 1H), 8.16-8.07 (m, 8H), 7.96-7.92 (m, 2H), 7.91 (s, 2H), 7.79 (ddd, J=8.4, 5.6, 1.6 Hz, 3H), 7.55 (ddd, J=8.2, 6.8, 1.2 Hz, 2H), 6.39 (s, 1H), 6.34 (s, 1H), 4.49 (t, J=5.0 Hz, 4H), 4.32 (t, J=5.5 Hz, 2H), 4.27 (dd, J=7.7, 4.8 Hz, 1H), 4.09 (dd, J=7.8, 4.5 Hz, 1H), 3.60 (t, J=6.4 Hz, 3H), 3.56-3.47 (m, 13H), 3.34 (t, J=5.9 Hz, 4H), 3.14 (q, J=5.9 Hz, 2H), 3.05 (ddd, J=8.6, 6.2, 4.4 Hz, 1H), 2.78 (dd, J=12.5, 5.1 Hz, 1H), 2.58-2.51 (m, 1H), 2.35 (t, J=6.4 Hz, 2H), 2.03 (t, J=7.5 Hz, 2H), 1.57 (ddt, J=12.3, 9.6, 6.0 Hz, 1H), 1.44 (ddt, J=13.9, 10.0, 6.2 Hz, 3H), 1.26 (h, J=8.1 Hz, 2H). ¹³C NMR (126 MHz, DMSO) δ172.60, 171.06, 167.59, 163.74, 163.16, 162.14, 152.76, 151.49, 147.46, 131.29, 127.19, 124.99, 123.15, 119.47, 115.65, 112.57, 95.47, 70.20, 70.14, 70.11, 69.98, 69.58, 67.95, 67.17, 65.61, 61.48, 59.64, 55.86, 40.22, 40.13, 40.05, 39.96, 39.88, 39.79, 38.87, 38.69, 36.49, 35.52, 28.63, 28.48, 25.70. HRMS (ESI-QTof) m/z: [M+H]⁺ calculated for C₅₂H₆₈N₁₁O₁₂S₂⁺: 1070.2330; found, 1070.4765, 535.7427.

### 3. Synthesis of PhenDC3-bio

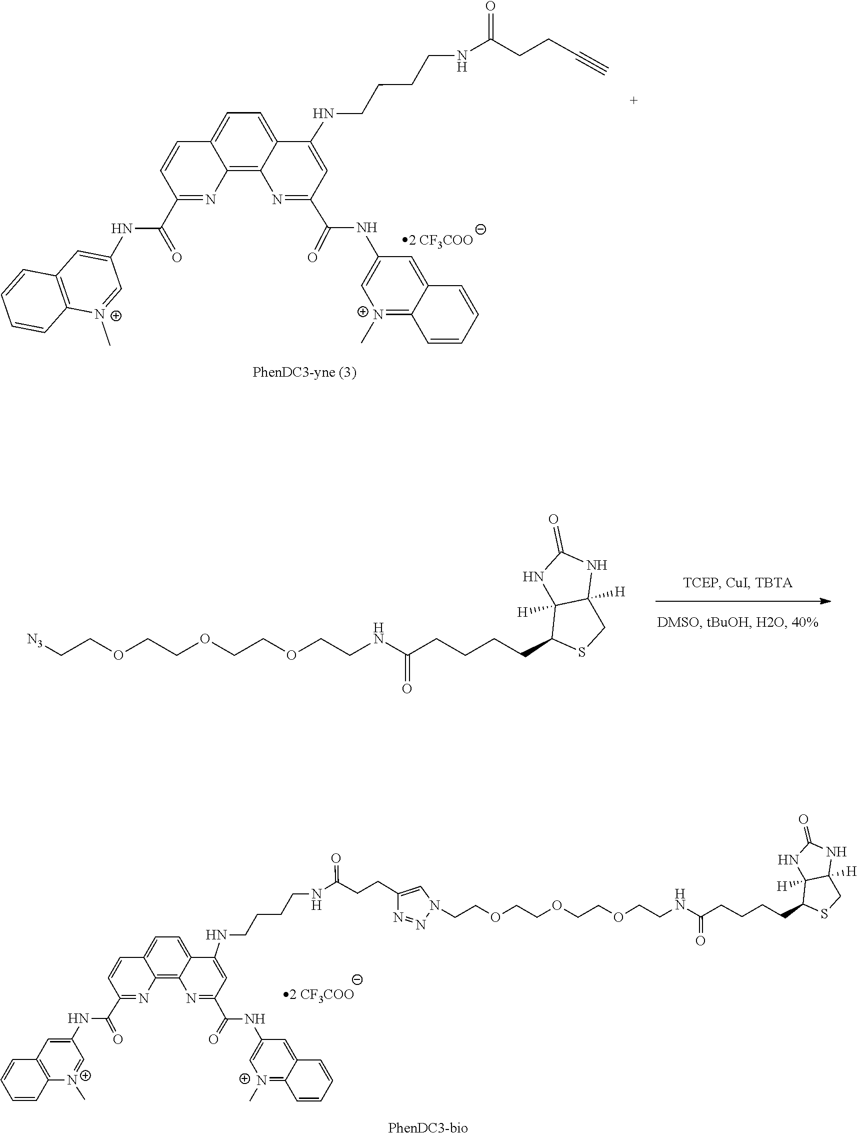 [SMILES: C#CCCC(=O)NCCCCNc1cc(C(=O)Nc2cc3ccccc3[n+](C)c2)nc2c1ccc1ccc(C(=O)Nc3cc4ccccc4[n+](C)c3)nc12 ; [N-]=[N+]=NCCOCCOCCOCCNC(=O)CCCC[C@@H]1SC[C@@H]2NC(=O)N[C@@H]21 ; C[n+]1cc(NC(=O)c2ccc3ccc4c(NCCCCNC(=O)CCc5cn(CCOCCOCCOCCNC(=O)CCCC[C@@H]6SC[C@@H]7NC(=O)N[C@@H]76)nn5)cc(C(=O)Nc5cc6ccccc6[n+](C)c5)nc4c3n2)cc2ccccc21]

PhenDC3-yne (3) was prepared by following reports by Lefebvre et at Angewandte Chemie International Edition, 56 (2017) 11365-11369). ¹H NMR (400 MHz, DMSO) δ12.15 (s, 2H), 10.30 (d, J=2.8 Hz, 2H), 9.88 (s, 2H), 8.87 (d, J=8.4 Hz, 1H), 8.68 (d, J=8.3 Hz, 1H), 8.62 (d, J=9.4 Hz, 1H), 8.56-8.47 (m, 4H), 8.25 (s, 2H), 8.21 (d, J=9.5 Hz, 1H), 8.07 (t, J=6.8 Hz, 3H), 7.98 (s, 1H), 7.70 (s, 1H), 4.73 (s, 3H), 4.72 (s, 3H), 3.51 (s, 2H), 3.18 (d, J=6.0 Hz, 2H), 2.75 (t, J=2.6 Hz, 1H), 2.39-2.34 (m, 2H), 2.28 (t, J=7.0 Hz, 2H), 1.80 (s, 2H), 1.62 (s, 2H). ¹³C NMR (101 MHz, DMSO) δ170.2, 164.2, 163.7, 152.5, 148.0, 145.8, 145.7, 145.8, 145.7, 138.8, 135.5, 135.4, 134.7, 134.5, 133.9, 133.8, 132.8, 131.0, 130.3, 130.0, 129.9, 129.2, 125.3, 122.9, 121.6, 119.4, 119.2, 99.7, 83.8, 71.3, 46.0, 42.6, 38.2, 34.3, 27.0, 25.1, 14.3; HRMS (ESI⁺) calculated for [C₄₅H₄₀N₈O₅F₃]⁺: 829.3068, m/z found: 829.3057.

PhenDC3-yne (7, 5.4 mg, 6.5 μmol) and Biotin-PEG3-azide (6 mg, 13 μmol) were suspended in 75 μL DMSO. Tris(2-carboxyethyl)phosphine (TCEP, 0.33 mmol, freshly prepared 100 mM solution in water) was added, followed by copper(II) sulphate pentahydrate (0.33 mmol, freshly prepared 10 mM in water). Tris(benzyltriazolylmethyl)amine (TBTA, 0.33 mmol, stock solution 40 mM in 1:1 water/tert-butanol mixture) was added to the solution and stirred for 3 hs at room temperature under argon. The mixture was purified using flash column chromatography (C18 column, gradient elution: water (0.1% (v/v) TFA) to MeCN (0.1% (v/v) TFA) over 30 min at a flow rate of 18 mL min⁻¹ Solvents were removed through freeze-drying to obtain the product as a yellow powder (PhenDC3-bio, 2.9 mg, 41%). HRMS (ESI-QTof) m/z: [M2+]⁺ calculated for C₆₁H₇₂N₁₄O₈S²⁺: 580.26836; found, 580.26768, 387.18184.

### 4. Synthesis of Dox-bio1

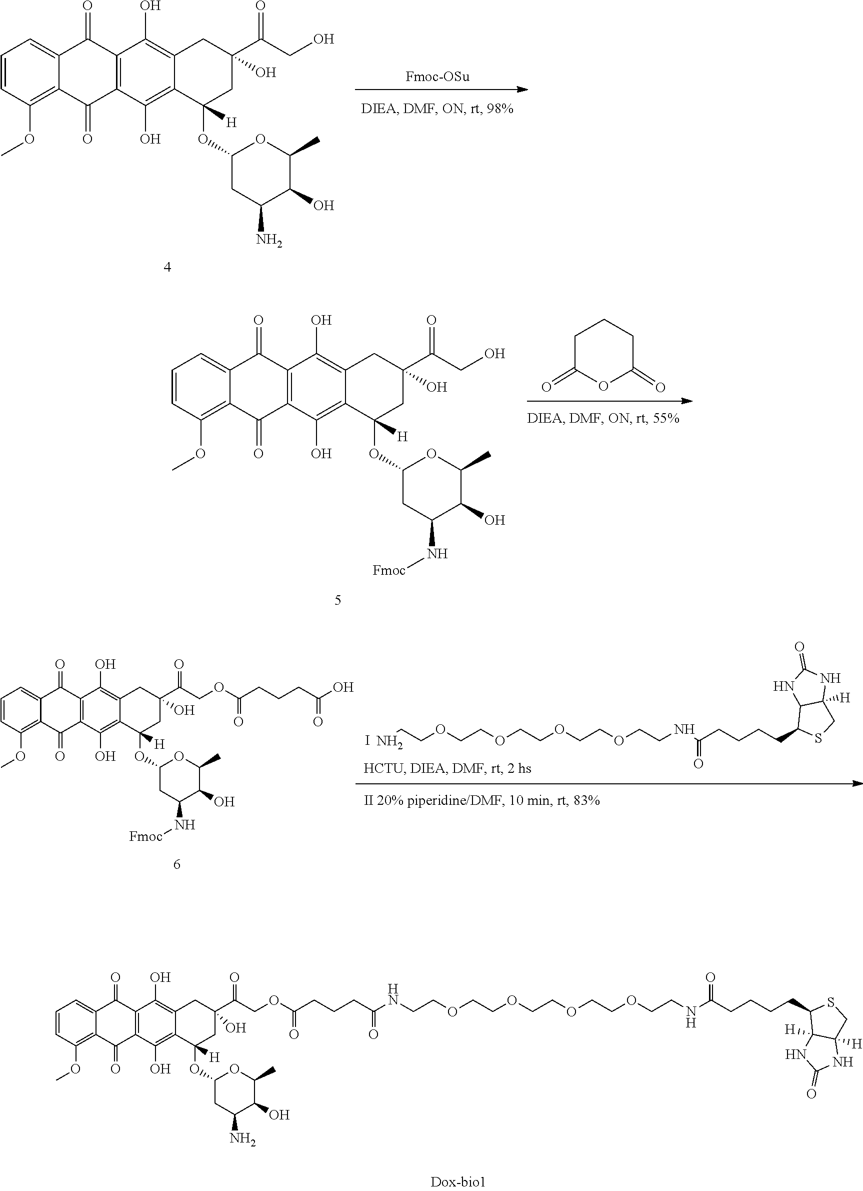

Following reports by Tjandra et al. J. Med Chem, 63 (2020) 2181-2193, doxorubicin·HCl (4, 29 mg, 0.05 μmol) was dissolved in 4 mL anhydrous DMF. Fmoc N-hydroxysuccinimide ester (Fmoc-OSu, 26 mg, 0.075 mmol) was dissolved in 1 mL DMF and then added to the solution with constant stirring. DIEA (60 μL, 0.34 mmol) was added dropwise to the mixture upon which a dark red solution formed. The mixture was stirred at room temperature under argon and protected from light. The reaction stopped after 4 hs. DMF was removed in vacuo, and the remaining red oil was added with 0.1% (v/v) aqueous trifluoroacetic acid to give red crystals and washed with cold Et₂O. The product doxorubicin-Fmoc (5) was then dried in vacuo and collected as red crystals (38 mg, 98%). This intermediate (5, 38 mg, 0.05 mmol) was redissolved in 5 mL anhydrous DMF and reacted with glutaric anhydride (53 mg, 0.25 mmol). DIEA (18 μL, 0.1 mmol) was added dropwise to the reaction mixture to form a dark red solution. The mixture was protected from light and stirred overnight at room temperature under nitrogen atmosphere. After 24 hs, the reaction mixture was concentrated, and the residual red oil was triturated with 0.1% (v/v) aqueous trifluoroacetic acid to produce a red solid powder. This crude product was purified using C18 flash column on a 254 nm and 200-300 nm UV detector with MeCN (B) and 0.1% formic acid in water (A) as eluent and obtained red powder (6, 24 mg, yield 55%). N-Fmoc-doxorubicin-O-hemiglutarate (6) (10 mg, 0.011 mmol) was dissolved in 0.5 ml anhydrous DMF. HCTU (5.6 mg, 13 μmol) and DIEA (3.8 μL, 22 μmol) were added to the solution. The mixture was stirred for 2 hs under argon atmosphere. The solvent was evaporated to dryness in vacuo. The intermediate was resuspended in 1 mL 20% piperidine/DMF (v/v) solution and stirred for 10 min at room temperature. The solvent was evaporated to dryness in vacuo. The product was purified using flash column chromatography (C18 column, gradient elution: water (0.1% (v/v) TFA) to MeCN (0.1% (v/v) TFA) over 30 min at a flow rate of 18 mL/min). Solvents were removed through freeze-drying to obtain the product as a red powder (Dox-bio1, 10.1 mg, yield 83%). ¹H NMR (500 MHz, DMSO) δ14.05 (s, 1H), 13.27 (s, 1H), 7.96-7.93 (m, 2H), 7.86 (t, J=5.6 Hz, 1H), 7.82 (t, J=5.7 Hz, 1H), 7.74 (d, J=5.3 Hz, 3H), 7.71-7.66 (m, 1H), 6.40 (s, 1H), 6.35 (s, 1H), 5.59 (s, 1H), 5.46 (d, J=6.3 Hz, 1H), 5.32-5.28 (m, 1H), 5.24 (d, J=17.8 Hz, 1H), 5.15 (d, J=17.8 Hz, 1H), 4.98 (dd, J=5.5, 2.9 Hz, 1H), 4.30 (dd, J=7.8, 5.0 Hz, 1H), 4.21 (q, J=6.3 Hz, 1H), 4.12 (ddd, J=7.8, 4.4, 1.9 Hz, 1H), 4.00 (s, 4H), 3.55 (d, J=5.6 Hz, 1H), 3.39 (q, J=5.9 Hz, 6H), 3.18 (p, J=6.0 Hz, 5H), 3.13-3.07 (m, 3H), 3.05 (s, 1H), 2.93 (d, J=18.2 Hz, 1H), 2.81 (dd, J=12.4, 5.1 Hz, 1H), 2.57 (d, J=12.4 Hz, 1H), 2.40 (t, J=7.5 Hz, 2H), 2.28 (d, J=14.4 Hz, 1H), 2.14 (t, J=7.4 Hz, 2H), 2.10 (d, J=5.6 Hz, 1H), 2.05 (t, J=7.4 Hz, 3H), 1.90 (td, J=12.7, 3.7 Hz, 1H), 1.77 (p, J=7.6 Hz, 3H), 1.69 (dd, J=12.2, 4.4 Hz, 1H), 1.60 (ddd, J=15.9, 10.1, 4.7 Hz, 1H), 1.54-1.39 (m, 4H), 1.29 (dt, J=15.7, 7.7 Hz, 3H), 1.19-1.14 (m, 5H). ¹³C NMR (126 MHz, DMSO) δ207.82, 186.71, 186.61, 172.13, 172.10, 171.54, 162.69, 160.86, 155.94, 154.44, 136.38, 135.10, 134.83, 133.85, 120.05, 119.85, 119.09, 110.86, 110.79, 99.27, 75.10, 69.78, 69.72, 69.55, 69.16, 69.10, 66.20, 66.14, 65.41, 61.02, 59.18, 56.66, 55.41, 46.66, 45.75, 39.69, 39.61, 39.52, 39.44, 38.45, 38.43, 36.11, 35.08, 34.13, 32.58, 31.87, 28.19, 28.03, 25.25, 20.65, 16.62, 8.64. HRMS (ESI-QTof) m/z: [M+H]⁺ calculated for C₅₂H₇₃N₅O₁₉S⁺: 1102.2160; found, 1102.4580.

### 5. Synthesis of Dox-bio2

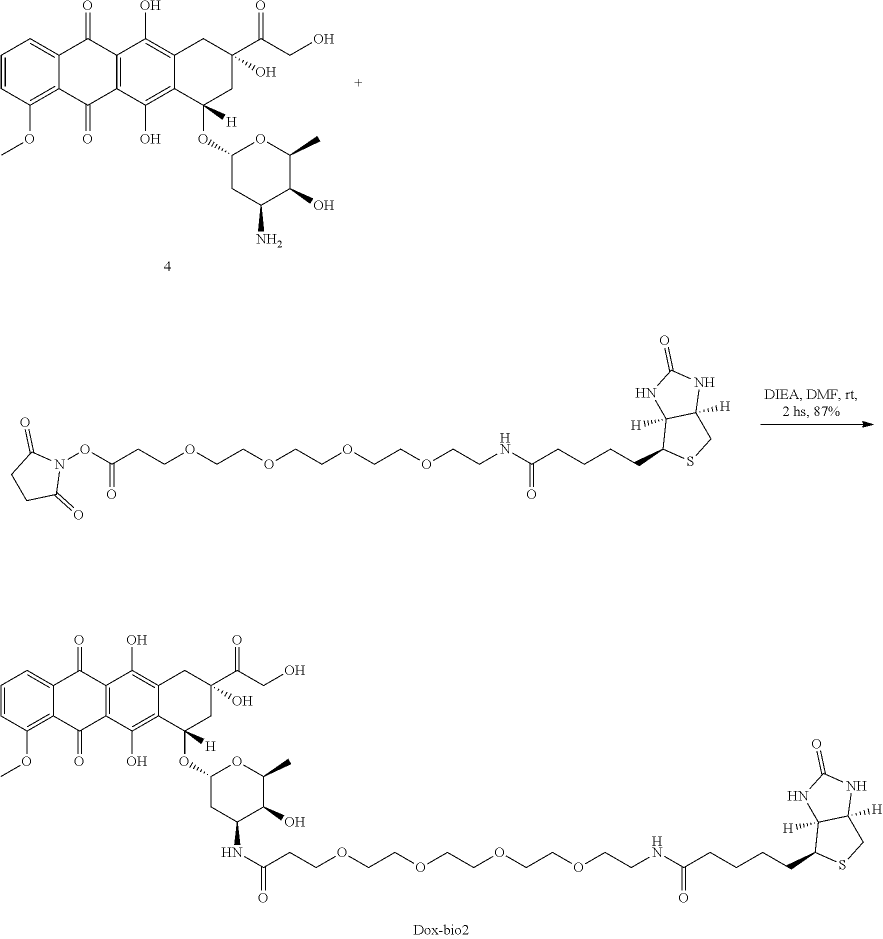 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1 ; O=C(CCCC[C@@H]1SC[C@@H]2NC(=O)N[C@@H]21)NCCOCCOCCOCCOCCC(=O)ON1C(=O)CCC1=O ; COc1cccc2c1C(=O)c1c(O)c3c(c(O)c1C2=O)C[C@@](O)(C(=O)CO)C[C@@H]3O[C@H]1C[C@H](NC(=O)CCOCCOCCOCCOCCNC(=O)CCCC[C@@H]2SC[C@@H]3NC(=O)N[C@@H]32)[C@H](O)[C@H](C)O1]

To a 5 mL flask, doxorubicin·HCl (4, 20 mg, 36 mmol) and biotin-PEG4-OSu (22 mg, 36 μmol) were dissolved in 1 mL anhydrous DMF. DIEA (7.1 μL, 55 μmol) was added, and the reaction was stirred for 2 hs in room temperature under argon. The solvent was evaporated to dryness in vacuo. The product was purified using flash column chromatography (C18 column, gradient elution: water (0.1% (v/v) TFA) to MeCN (0.1% (v/v) TFA) over 30 min at a flow rate of 18 mL/min). Solvents were removed through freeze-drying to obtain the product as a red powder (Dox-bio2, 32 mg, yield 87%). ¹H NMR (500 MHz, DMSO) δ14.04 (s, 1H), 13.28 (s, 1H), 7.94-7.87 (m, 2H), 7.79 (t, J=5.7 Hz, 1H), 7.69-7.61 (m, 1H), 7.55 (d, J=8.1 Hz, 1H), 6.39 (s, 1H), 6.33 (s, 1H), 5.45 (s, 1H), 5.21 (d, J=3.7 Hz, 1H), 4.94 (dd, J=5.6, 3.5 Hz, 1H), 4.55 (s, 2H), 4.31-4.25 (m, 1H), 4.14 (q, J=6.7 Hz, 1H), 4.10 (dd, J=7.8, 4.4 Hz, 1H), 3.98 (s, 3H), 3.95 (s, 1H), 3.57 (s, 4H), 3.51 (td, J=6.6, 1.7 Hz, 4H), 3.42 (ddt, J=8.5, 5.5, 3.6 Hz, 6H), 3.38-3.30 (m, 4H), 3.14 (q, J=5.9 Hz, 2H), 3.10-2.99 (m, 1H), 2.97 (d, J=5.0 Hz, 2H), 2.79 (dd, J=12.4, 5.1 Hz, 1H), 2.61-2.52 (m, 1H), 2.41-2.08 (m, 5H), 2.03 (t, J=7.4 Hz, 2H), 1.81 (td, J=12.9, 4.0 Hz, 1H), 1.58 (ddd, J=20.6, 10.7, 6.1 Hz, 1H), 1.52-1.37 (m, 4H), 1.27 (dq, J=14.3, 7.6 Hz, 2H), 1.11 (d, J=6.5 Hz, 3H). ¹³C NMR (126 MHz, DMSO) δ207.82, 186.71, 186.61, 172.13, 172.10, 171.54, 162.69, 160.86, 157.75, 157.51, 155.94, 154.44, 136.38, 135.10, 134.83, 133.85, 120.05, 119.85, 119.09, 110.86, 110.79, 99.27, 75.10, 69.78, 69.72, 69.55, 69.16, 69.10, 66.20, 66.14, 65.41, 61.02, 59.18, 56.66, 55.41, 46.66, 45.75, 38.45, 38.43, 36.11, 35.08, 34.13, 32.58, 31.87, 28.19, 28.03, 25.25, 20.65, 16.62, 8.64. HRMS (ESI-QTof) m/z: [M+H]⁺ calculated for C₄₈H₆₅N₄O₁₈S⁺: 1017.1100; found, 1017.4043.

### Cell Culture and Compound Treatment

Mycoplasma-free human chronic myelogenous leukemia K562 cells (RRID:CVCL_0004) derived from a 53-year-old female were purchased from ATCC and cultured in RPMI 1640 (Gibco, 21875034) supplemented with 10% heat-inactivated fetal bovine serum (FBS, Gibco, A3840401). Human bone osteosarcoma epithelial U-2 OS cells (RRID:CVCL_0042) derived from a moderately differentiated sarcoma of the tibia of a 15-year-old female osteosarcoma were obtained from ATCC and cultured in DMEM (Gibco, 41966029) supplemented with 10% FBS. Both cell lines were grown in accordance with ENCODE cell culture protocols and periodically tested for mycoplasma contamination and identity confirmed by short tandem repeat (STR) typing. HDAC inhibitors (HDACi) were prepared at 10 mM as stock solution in DMSO. Cells were treated with HDACi dissolved to 1 μM final concentration or an equal concentration of vehicle (0.1% DMSO) for 72 hs.

### CUT&Tag

Recombinant pA-Tn5 and transposome assembly with DNA adapters have been described in detail elsewhere (W. W. I. Hui, et al. Scientific Reports, 11 (2021) 1930). BRD4 CUT&Tag was performed as described before (H.S. Kaya-Okur, et al. Nat. Protoc., 15 (2020) e23641). Briefly, cells were incubated with activated concanavalin A-coated magnetic beads (Bangs Labs, BP531). The bead-bound cells were permeabilized and incubated with anti-BRD4 (E2A7X) rabbit antibody (Cell Signalling Technology, 13440), followed by rabbit anti-mouse IgG antibody (Antibodies-Online, ABIN101961, RRID:AB_10775589). Diluted pA-Tn5 adapter complex was then added followed by the tagmentation reaction. Extracted DNA fragments were used for library preparation and Illumina sequencing.

### Chemmap

Cell preparation. U-2 OS cells were detached using accutase and immediately quenched using complete culture media. U-2 OS and K562 suspension cells were harvested by centrifugation and fixed in 0.1% formaldehyde (Thermo Scientific, 28906) in PBS for 2 min at room temperature, followed by quenching with glycine to a final concentration of 75 mM. Fixed cells were collected by centrifugation at 600×g for 4 min, followed by resuspension in cold wash buffer (20 mM HEPES pH7.5, 150 mM NaCl and 0.5 mM spermidine (Sigma, S0266)) in nuclease-free water supplemented with a complete Protease Inhibitor, EDTA-free (Sigma, 11873580001). (Note: For Chemmap of G4 ligands NaCl was replaced by equivalent concentrations of KCl in all buffers to maintain G4 stability. For optimal results, the concentration of cells needs to be adjusted based on target abundance and relative affinity of the probes. We employed cells at 1500 cells/μL (JQ1-bio) and 6000 cells/μL (G4 ligands and biotinylated doxorubicin))

Probe-1^{st} Ab complex assembly. Compound stock solution in DMSO was diluted to 10 μM in antibody buffer (2 mM EDTA, 0.1% BSA (Sigma, A8577), 0.05% digitonin (EMD Millipore, 300410) in wash buffer). For 5 samples, 20 μL probe solution (10 μM) and 16.7 μL anti-biotin (D5A7) Rabbit mAb (Cell Signaling Technology, 5597, concentration is ˜10 μM) were added to 200 μL antibody buffer and incubated on ice for 1 h to perform a complex at high concentration (probe in excess 1.2:1 to avoid non-specific antibody binding).

Next, 300 μL antibody buffer were added to the probe-1^{st} Ab complexes solution for a final small molecule concentration of 0.4 μM.

Bead capture. For 5 samples, 50 μL (JQ1-bio) or 75 μL (G4 ligands and biotinylated Doxorubicin) concanavalin A beads (Bangs Labs, BP531) were washed twice in 1 mL binding buffer (20 mM HEPES pH 7.5, 10 mM KCl, 1 mM CaCl₂, 1 mM MnCl₂ in nuclease-free water) and resuspended in 75 μL binding buffer. 100 L of cell suspension were incubated with 10 μL pre-washed concanavalin A beads at 25° C. for 10 min at 600 rpm. Beads-bound cells were gently washed twice with wash buffer, before resuspending in 100 μL probe-1^{st} Ab solution and incubating at 4° C. overnight at 600 rpm.

2^{nd} Ab-Tn5 transposome complexes assembly. For 5 samples, 2.5 μL 2^{nd} Ab (Antibodies-Online, ABIN101961, RRID:AB_10775589) and 5 μL pA-Tn5 were added to 200 μL Dig-300 buffer (20 mM HEPES pH 7.5, 300 mM NaCl, 0.5 mM spermidine, 0.01% digitonin in nuclease-free water supplemented with Complete Protease Inhibitor, EDTA-free) and incubated on ice for 1 h. 300 μL antibody buffer were added to the 2^{nd}-Tn5 complexes solution. (2^{nd} Ab-Tn5 complexes at a ratio of 2:1).

Tagmentation. Cells were washed 3× with 500 μL Dig-wash buffer (0.05% digitonin in wash buffer) and resuspended in 100 μL 2^{nd} Ab-Tn5 transposome solution and incubated at 25° C. for 1 h at 600 rpm. Cells were then washed 3× in 500 μL Dig-300 buffer before incubation in 300 μL tagmentation buffer (10 mM MgCl₂ in Dig-300 buffer) at 37° C. for 1 h at 600 rpm.

DNA extraction. After tagmentation, cells were washed twice with 500 μL TAPS wash buffer (10 mM TAPS (Alfa Aesar, J63268.AE), 0.2 mM EDTA in nuclease-free water). 150 μL of extraction buffer (0.5 mg/mL proteinase K (Thermo Scientific, EO0491), 0.5% SDS in 10 mM Tris-HCl pH8.0) were added, vortexed and incubated at 55° C. for 1 h at 800 rpm. Next, 100 μL phenol-chloroform-isoamyl alcohol (Invitrogen, 15593049) was added and mixed. The mixture was transferred to MaXtract High Density Tube (QIAGEN, 129046) and centrifuged at room temperature for 3 min at 16,000×g. 150 μL chloroform were added to the top aqueous phase, mixed by inverting the phase-lock tubes and centrifuged at room temperature for 3 min at 16,000×g. The top aqueous layer was transferred to a 1.5 mL DNA Lo-bind tube (Eppendorf, 022431021). 6 μL 5 M NaCl and 375 μL cold ethanol were added, mixed and incubated at −20° C. overnight. Samples were centrifuged at 4° C. for 30 min at 21,130×g. The supernatant was carefully poured off and the DNA pellet rinsed with 1 mL cold 100% ethanol followed by centrifugation at 4° C. for 2 min at 21,130×g. After pouring off the wash and draining the residual liquid with paper towel, the pellet was left to air-dry. Finally, the pellet was resuspended in 25 μL elution buffer 1 (10 mM Tris-HCl pH8, 1 mM EDTA, 1/400 RNAseA (Thermo Scientific, EN0531) in nuclease-free water) by vortexing and incubating at 37° C. for 10 min at 800 rpm. Library preparation. In a 0.2 mL PCR tube, 25 μL NEBNext HiFi 2×PCR master mix (NEB, M0541), 2 μL of 10 μM Ad1 [J. D. Buenrostro, et al., Nature, 523 (2015) 486-490], 2 μL of 10 μM Ad2 [J. D. Buenrostro, et al] and 21 μL tagmented DNA were added and subjected to PCR (72° C. for 5 min, 98° C. for 30 s, followed by 10 cycles of 98° C. for 10 s and 63° C. for 10 s, and one cycle of 72° C. for 1 min). Libraries were purified using 1.3×ratio (65 μL) Ampure XP beads (Beckman Coulter, A63882). After 10 min room temperature incubation, bead-bound DNA was washed twice with 80% ethanol and libraries eluted with 25 μL 10 mM Tris-HCl for 5 min at room temperature.

### Library Sequencing.

Library size and concentration were measured using a TapeStation HSD1000 ScreenTape (Agilent, 5067-5584). Libraries were balanced and pooled for size selection using Ampure XP beads. 0.4×ratio of Ampure XP beads were added to pooled libraries and the supernatant transferred to a new tube after 15 min at room temperature. 1.3 ratio of Ampure XP beads were then added to the supernatant and incubated for 15 min at room temperature. Beads were washed twice with 80% ethanol and libraires eluted in 40 μL 10 mM Tris-HCl. Libraries were sequenced on a NextSeq 500 sequencer (Illumina) with a paired-end format of 36 bp×2 using the High Output kit (Illumina, FC-404-2005).

### FRET Melting Assay.

400 nM FAM-TAMRA dual-labelled oligonucleotides (Biomers) were annealed in assay buffer (60 mM potassium cacodylate, pH=7.4) at 95° C. for 5 min followed by gradually cooling to 20° C. A series of probe concentrations were prepared in a 8-well strip tube: 150 μL of 6 μM ligand in assay buffer was prepared as the initial concentration. Subsequent serial dilutions were made by adding 100 μL of probe solutions to 50 μL of assay buffer, resulting in 12 concentrations including a control (1% DMSO). 25 μL per solutions were transferred to 96-well plate, followed by adding 25 μL of annealed oligonucleotide solutions to each well. The plate was then sealed with an adhesive transparent cover and shaken gently for 10 min at room temperature. Measurements of restoring FAM signal were recorded on a Bio-Rad CFX96 Touch Real-Time PCR Detection System employing a temperature gradient from 25° C. to 95° C. at 0.5° C./min. Melting temperatures were determined by the first derivative maxima of relative fluorescence unit (RFU) value against time, and ΔTₘ was calculated by baseline correction of melting temperatures subtracting control group. A one-site binding model in GraphPad Prism 7 was utilised to fit FRET Tₘ curves. Mean was calculated from two replicates.

### Cell Imaging

˜40,000 U-2 OS cells diluted in 1 ml DMEM medium supplemented with 10% FBS were plated in 12-well plate. After 18 hs incubation, live cells were treated for 6 hs with doxorubicin (1 μM), Dox-bio1 (1 μM), Dox-bio2 (1 μM) and 0.1% DMSO (control) respectively in fresh medium. Then cells were washed twice with PBS and lightly fixed with 1% formaldehyde for 2 min. Cells were then incubated with Hoechst 33342 for 10 min at 37° C. to label the cell nuclei. Imaging was captured in EVOS M5000. Blue-light filter cube with wavelength of Ex 357/44 and Em 447/60 was used for visualization of nuclei, and red-light filter cube with wavelength of Ex 531/40 and Em 593/40 was used for visualization of doxorubicin and its derivatives.

### Bioinformatic Data Processing

1. Data demultiplexing and de-duplication. Illumina sequencing paired end output files were demultiplexed using demuxIllumina version 3.0.9 using the flags; -c-d-i-e-t 1-r 0.01-R-I 9. The resulting fq.gz files underwent sequencing quality control using FastQC v0.11.8, and their summary was visualised by MultiQC v1.11. Bases with a quality score below 20 were trimmed from both reads using cutadapt (cutadapt-q 20). Fastq files were aligned to the combined hg38 and E. coli genomes using bwa 0.7.17-r1188 with only reads in the whitelisted regions of hg38 continuing the process pipeline. Duplicates were removed using Picard version 2.20.3 (Picard MarkDuplicates) and peaks were called using seacr version 1.3 at the top 1% and 5% of AUC without input control using both the relaxed and stringent criteria, and deduplicated bam files sorted and indexed.
2. Peak calling. Deduplicated bam files were transformed into bedpe files (bedtools bamtobed-bedpe) and then only fragments with size below 1000 bp (awk ‘{if ($1==$4 && $6-$2<1000) print $0}’) were retained. Next, coverage of <1000 bp fragments across human genome was computed using bedtools genomecov and reported in bedgraph format. SEACR was then used to identify regions of local enrichments. SEACR stringent search was performed to select top 5% peaks based on total signal within peaks (SEACR_1.3 $bdg_1000 0.05 non stringent). Peaks with a minimum coverage of eight reads were kept for further analysis.
3. Consensus regions and reference comparisons. Different thresholds were applied to resulting in peak files with the minimum total signal in peak coordinates at 5 8 and 10. For each threshold the overlap between the 5 technical replicates is calculated with intervene tools (venn upset and pairwise) and a series of .bed files containing at least 1 (union of all technical replicates) to 5 (common among all technical replicates) are created using multiIntersectBed with-wa wb flags of bedtools v.2.30.0. Intersection between biological replicates can follow the same pipeline. This classification of peaks allows their quantification and ranking according to the normalised (cpm) signal strength as well as the reproducibility of the peak.
   Assessment of G4 Enrichment Quality using Chemmap-qPCR

The G4 enrichment by Chemmap-qPCR was assessed by quantifying the relative enrichment of consistently enriched G4 DNA regions over background regions. For Chemmap-qPCR, a Chemmap 10×PCR sample was used. DNA concentration and size distribution were checked with an Agilent HS D1000 Tapestation and normalized to the same amount for qPCR.

PCR reaction condition: a master mix was made for each reaction. Chemmap samples were diluted 1 in 10. 5 μl of a 2×premix SYBRgreen qPCR mixture plus 2.5 μl primer mix was added to each well, and then 2.5 μl diluted Chemmap sample was added. A roller was used to add the plate seal, and the plate was spun (1 min 1000 rpm) before running qPCR. qPCR was performed with 40-45 cycles. RPA3, and MAZ were used as positive control and ESR1 and TMCC1 as negative control. Primers used include the following;

| Primer name | Sequence (5′-3′) |
|---|---|
| i7_primer_F | AAGCAGAAGACGGCATACGAG |
| i5_primer_F | ACGGCGACCACCGAGATCTAC |
| RPA3 forward | CGG AAG TTG ACA GAT ACA GGG |
| Reverse | GAT CGC AGA AAG GTA GTC TCA G |
| MAZ forward | ACT CAG CGC AGG ATT GTA AAT A |
| Reverse | CCT CAT GCT TCG GCT TCC |
| ESR1 forward | GAA ACA GCC CCA AAT CTC AA |
| Reverse | TTG TAG CCA GCA AGC AAA TG |
| TMCC1 forward | GTG GTA CAC TGC CTA CAG TAT T |
| Reverse | GTA TAA CGC CTG GGC TAT GT |

### Statistical Analyses

Data are presented as mean ±s.d. The sample sizes (n) in the figure legends indicate the number of replicates in each experiment and are provided in the corresponding figure legends. The peak or gene size (N) in the heat maps indicates the number of peaks or genes included. Statistical analysis in related figures, was performed by unpaired Student's t-tests, and the P values were denoted in each figure.

### Results

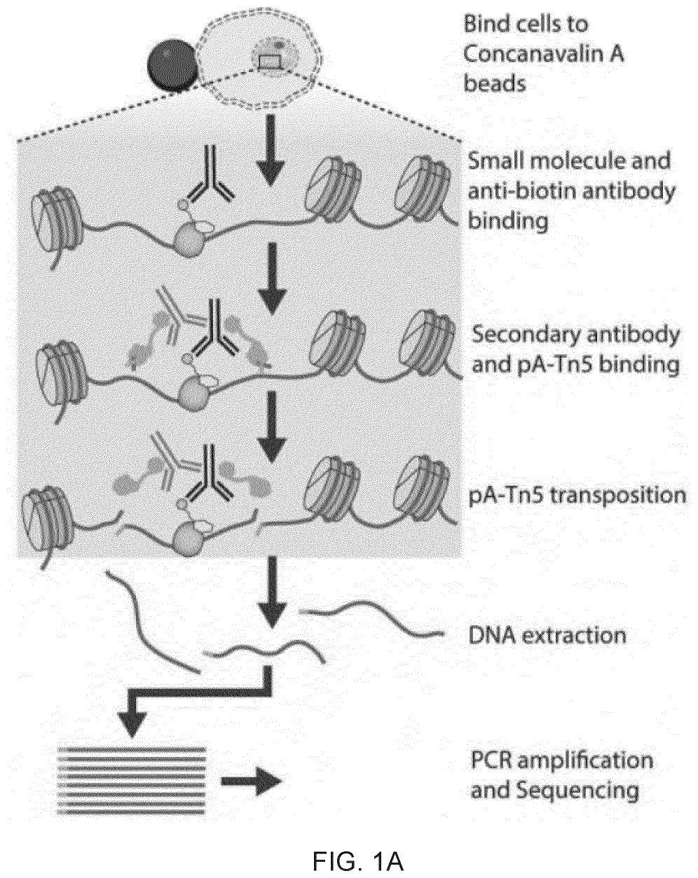

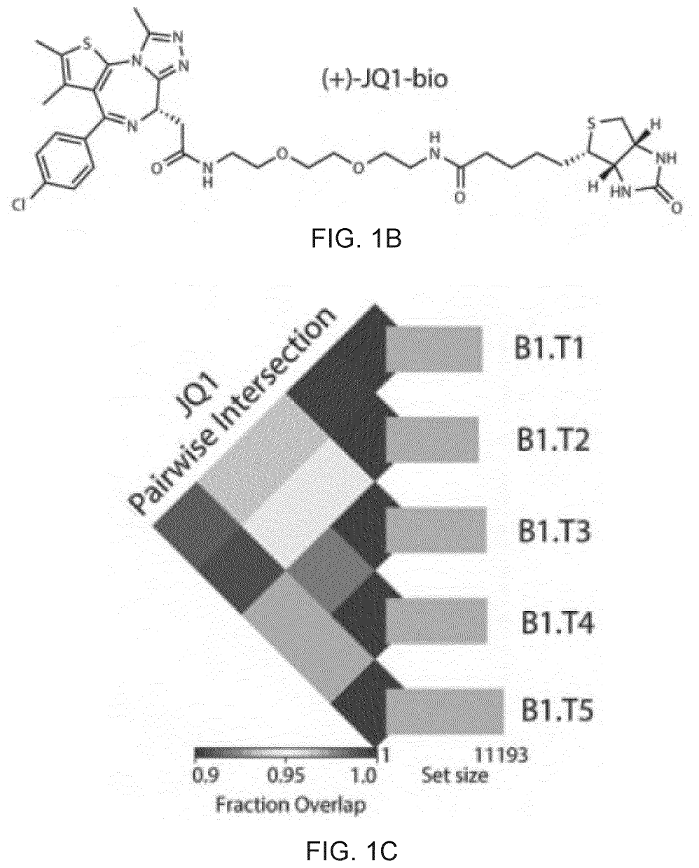

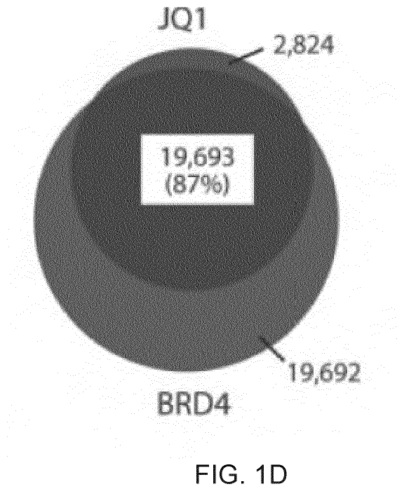

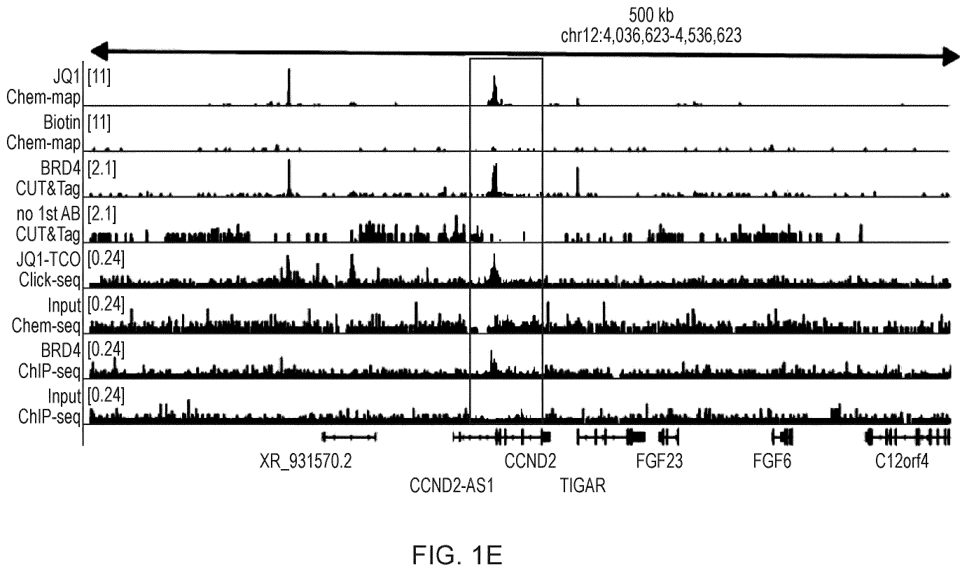

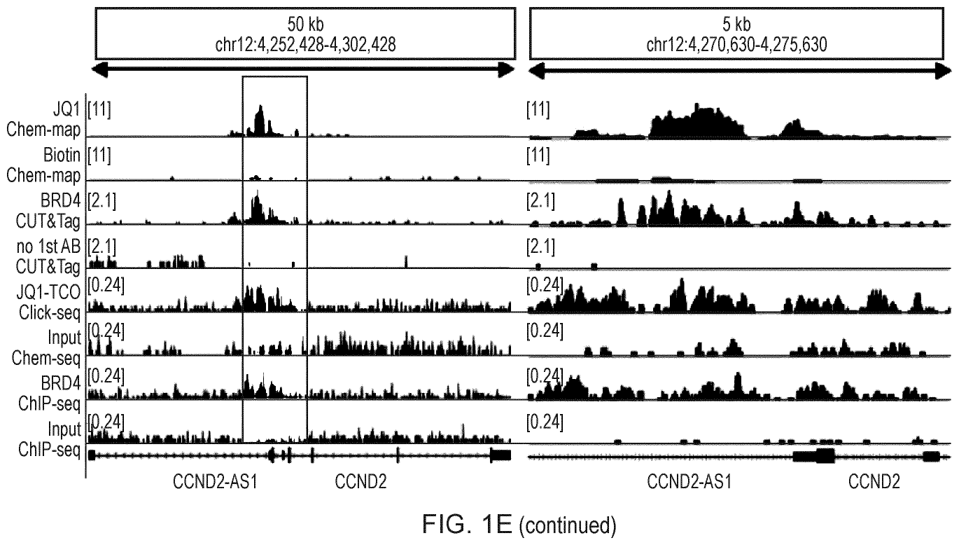

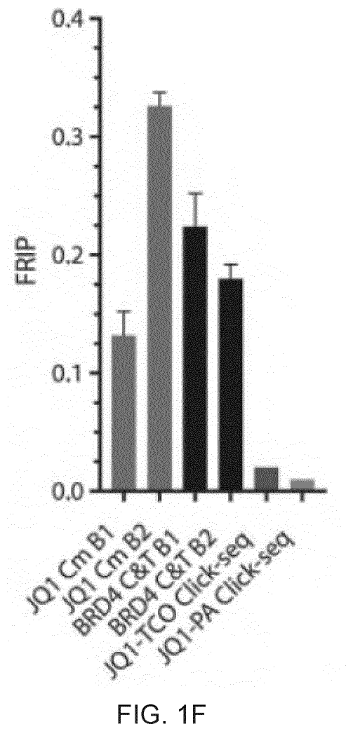

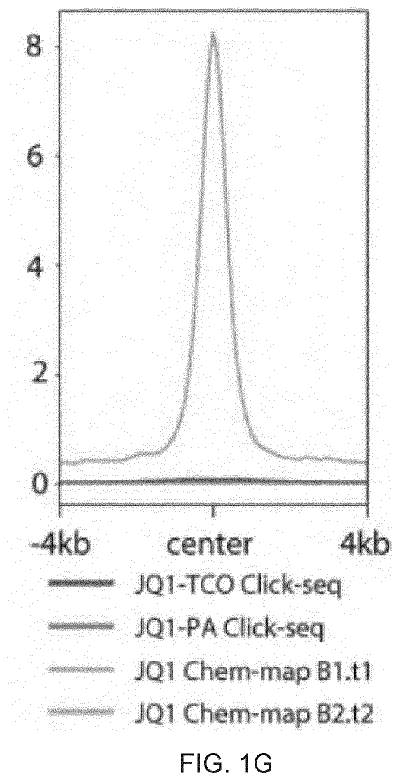

Chemmap is based on the introduction of an affinity tag to the small molecule. When the small molecule accesses its binding sites in the nucleus, the antibody that recognises the tag in small molecule-antibody precomplex can be used to recruit a secondary antibody pre-loaded with synthetic sequencing adapters and the transposase Tn5, which results in the insertion of sequencing adapters proximal to where the small molecule is bound in situ (FIG. 1A).

The recruitment of Tn5 to proteins bound to chromatin in lysate chromatin and in permeabilized cells in this way were exemplified by TAM-ChIP (Active Motif, M. Tedesco, et al., Nat. Biotechnol., 40 (2022) 235-244.) and CUT&Tag (9). Upon extraction of the DNA the adapted fragments that were in the proximity of the small molecule binding site can be selectively amplified by sequencing and then mapped by alignment to the genome. To validate Chemmap approach and compare the signal quality to established methods, we synthesised the biotinylated molecule JQ1 that had previously been mapped by Chem-Seq (FIG. 1B) (3, 5).

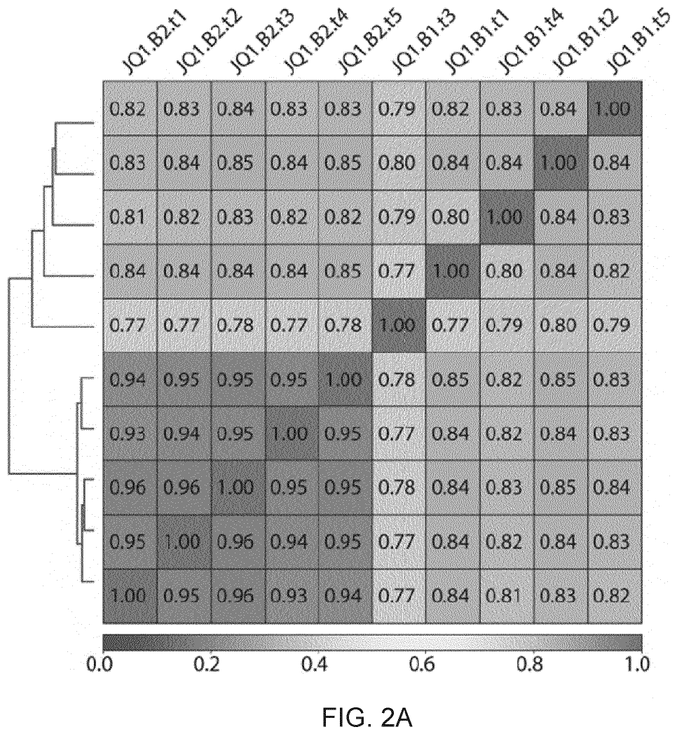

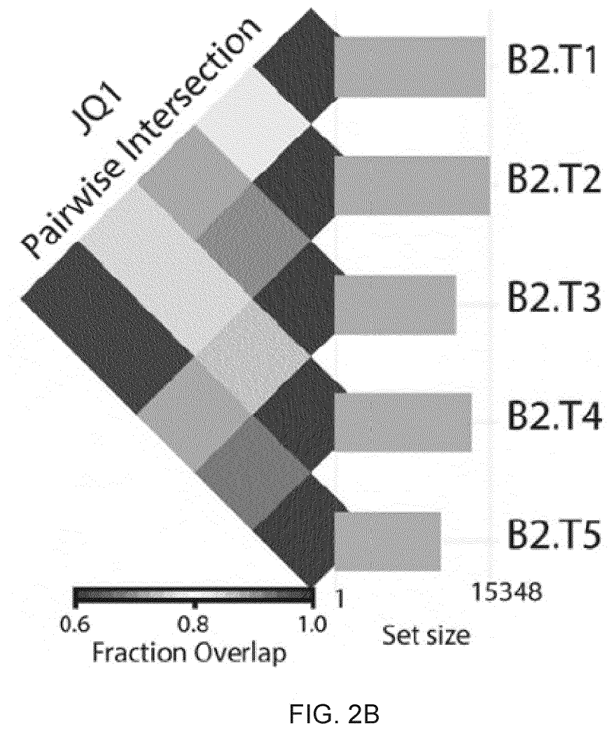

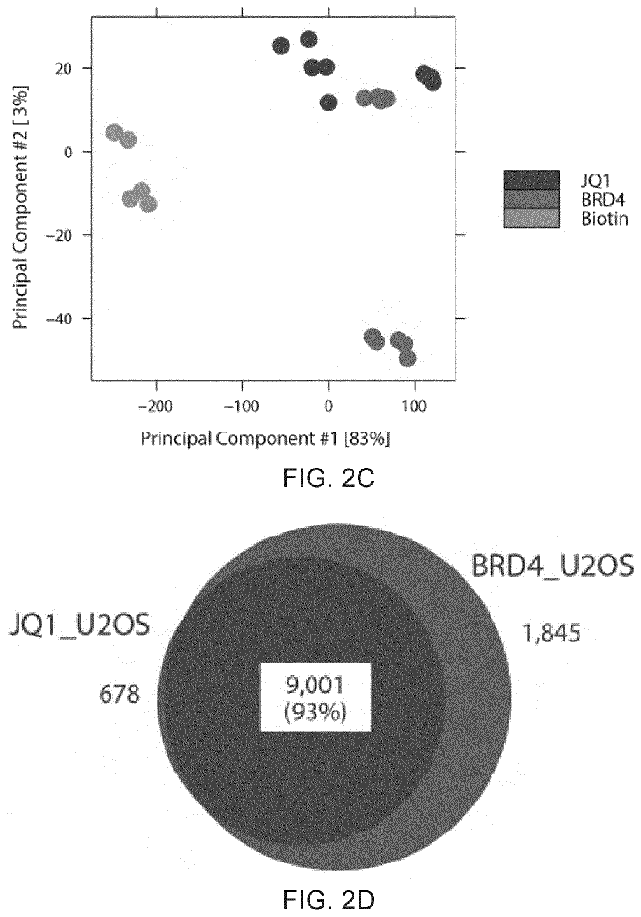

JQ1 is a well-characterised inhibitor of the BET family of bromodomain proteins with high binding affinity (10). In parallel, we also mapped the genome-wide binding sites of its main target BRD4 using a specific antibody in CUT&Tag (9). Mapping experiments with JQ1-bio were performed in 150,000 human leukaemia K562 cells performing two biological and five technical replicates to assess robustness and reproducibility of our approach. In each experiment we observed around 10,000 JQ1 binding sites with excellent reproducibility across both technical and biological replicates (rₛ>0.9, see FIG. 1C and 2A,B). Next, we compared sets of high confidence binding sites (see methods) from JQ1 Chemmap and BRD4 CUT&Tag finding that 93% of JQ1 peaks overlap with BRD4 binding sites. We investigated BRD4 binding sites that did not overlap with JQ1 high confidence peak sites, and after minor optimizing peak calling parameters based on the binding affinity difference of the two probes, it resulted in higher coverage of BRD4 binding sites (84%) with JQ1 (FIG. 2C). Similarly, differential binding analysis revealed a strong overlap of JQ1 and BRD4 binding sites (1,213/45,667 differential sites, 2.7%) (FIG. 2D). Moreover, principal components analysis (PCA) confirmed that JQ1 and BRD4 data cluster together and are distinctly away from the biotin control (FIG. 2E). Overall, this suggests that Chemmap confidently recapitulates JQ1 binding profile over its protein target and the observed differences are mostly due to non-optimal peak calling thresholds.

To assess the signal quality of Chemmap, we compared our findings to published JQ1 Click-Chem-seq data in K562 cells revealing substantially improved signal-to-noise in Chemmap (FIG. 1E)(5). To quantitatively compare methods, we plotted the average read counts for Chemmap and Click-Chem-seq around the highest confidence BRD4 binding sites (7,772 peaks present in all replicates) obtained by BRD4 CUT&Tag. Despite using two orders of magnitude lower cell numbers, we found that Chemmap provided ˜150-fold higher signal accumulation compared to Click-Chem-seq (maximum mean read count 8.17 cpm and 0.05 cpm, respectively) (FIG. 1F).

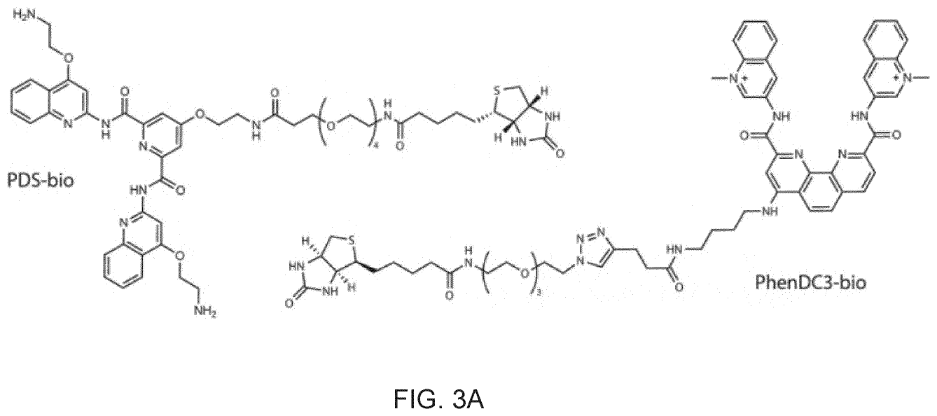

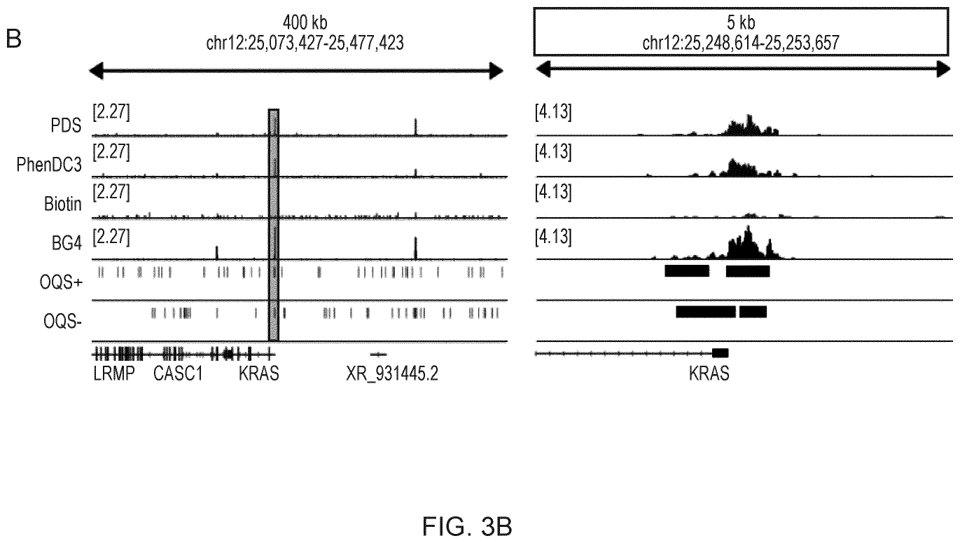

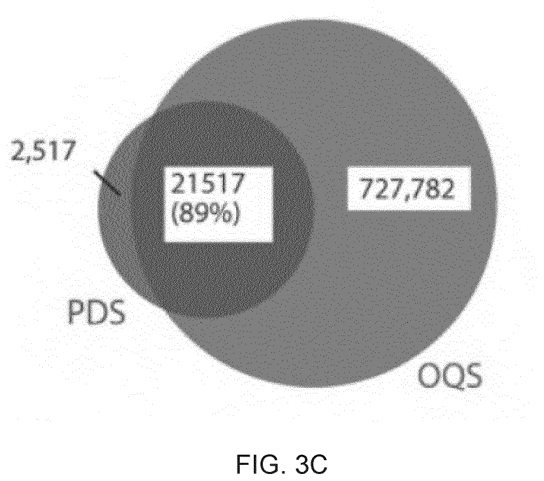

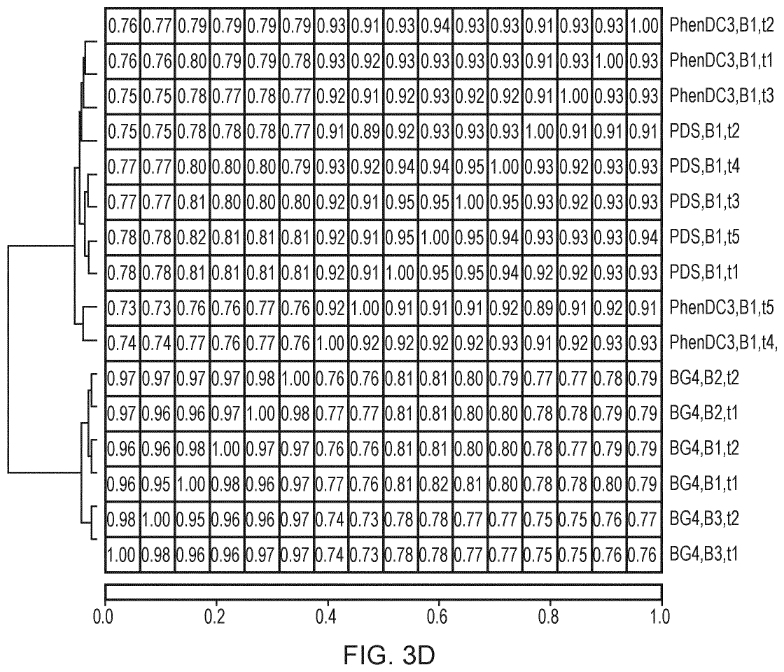

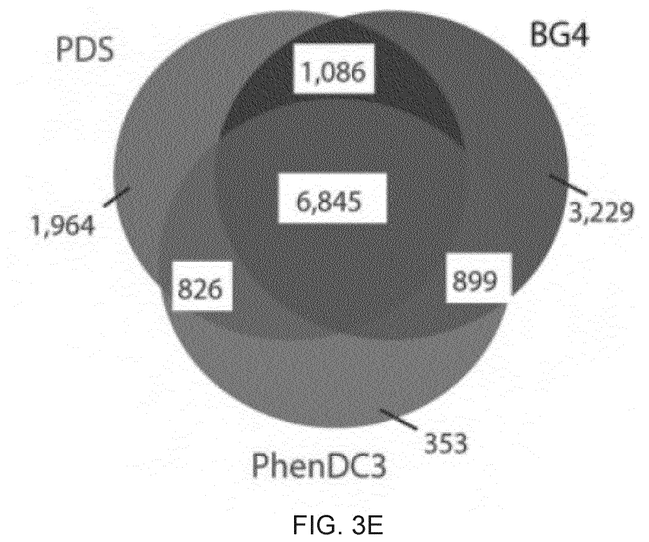

Having validated the method with a known protein inhibitor we then sought to map the binding sites of two widely used G-quadruplex (G4) targeted molecules, PDS and PhenDC3, which was impossible previously (FIG. 3a) (11, 12). G4s are four stranded structures that can form in G-rich DNA sequences. They have been implicated in gene regulation and are being considered as potential drug targets to treat cancers (13). Small molecules that bind to G4s can interfere with transcription and the replication fork, and by doing so cause DNA damage.

G4s themselves have only been recently mapped in chromatin using specific antibodies BG4 (14). Due to a lack of understanding on how BG4 recognize its binding targets and potential off-targets, there is a critical need to use a strikingly alternative method to cross validate the G4 topography in cells. Also, with the increasing numbers of study utilizing G4 ligand in preclinical and clinical research, we need to justify that these chemicals can indeed bind its target in situ. G4 ligand interacts with the plenary G-quartet surface of G4 via π-π stacking in crystal structure and has moderate binding affinity at μM level (15). However, it has not been possible to directly map the binding sites of such small molecules in the nuclei despite many attempts. There was limited success by enrichment sequencing via pulldown that only provided evidence of binding at telomeric repeats (16). Perhaps the most compelling evidence of target engagement at G4 loci has arisen from indirect methods such as mapping the sites of strand breaks induced as a downstream consequence of small molecule binding events (17). One of the main challenges in mapping DNA-small molecule interactions is dissociation of the ligand from the DNA target during washing steps leading to low recovery and bad signal-to-noise ratio.

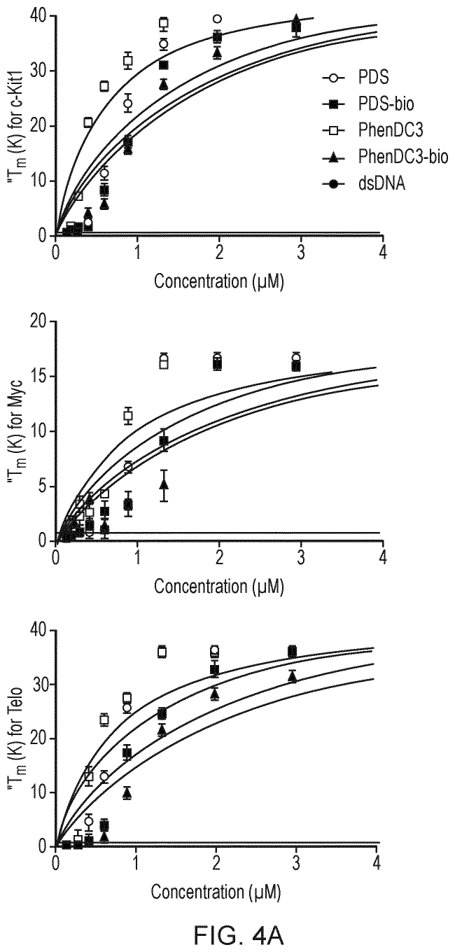

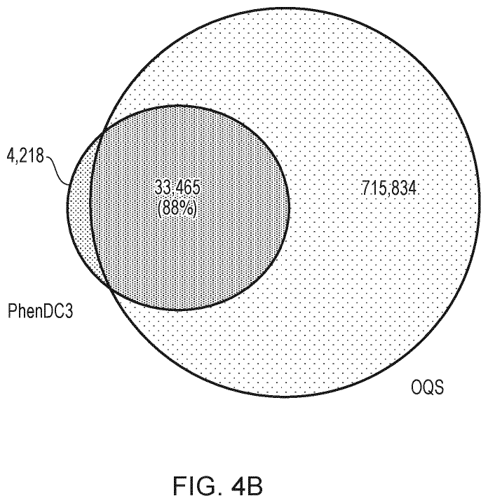

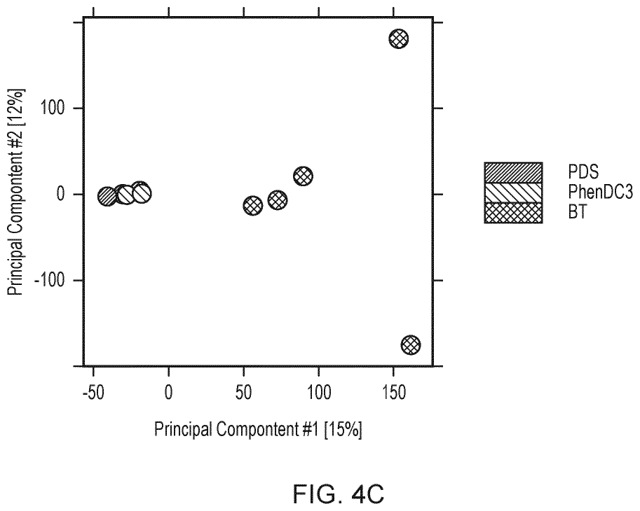

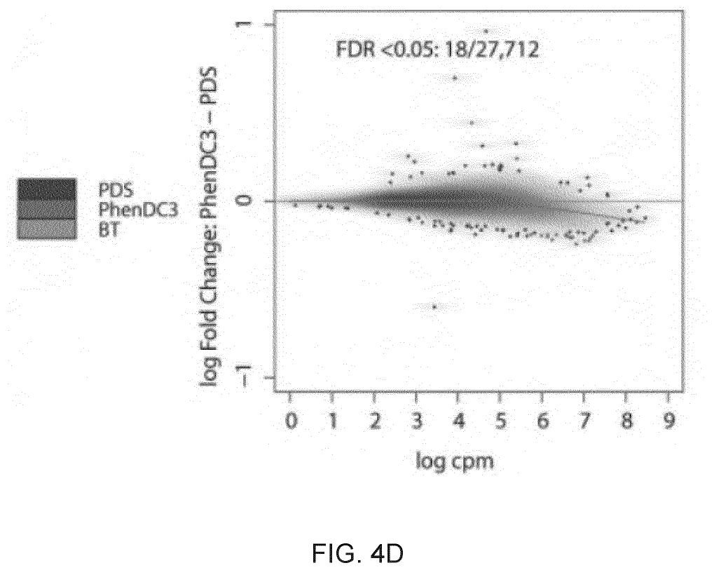

Our previous attempts to map G4 ligands using conventional Chem-seq did not work most likely due to the relatively low affinity of the small molecules (16). Given the substantially improved sensitivity obtained with JQ1 Chemmap, we reasoned that a transposome-based method in permeabilised cells (FIG. 1a) would necessitate the lifetime of the DNA-small molecule interaction merely be long enough to allow for catalytic insertion of the adapters to mark and select the binding sites. Ligands PDS-biotin and PhenDC3-azide were synthesised and evaluated in biophysical assay as reported previously, and PhenDC3-biotin was conjugated by copper assisted click reaction (16, 18). We inserted a flexible, long PEG4 linker in both probes to minimize binding perturbation and steric hindrance during antibody binding and DNA fragmentation (FIG. 3A). A fluorescence resonance energy transfer (FRET)-melting assay was employed to validate binding of the tagged G4 ligands to different G4 oligomers in vitro (FIG. 4A). We employed both probes in our established Chemmap protocol but using 600,000 K562 cells per experiment to compensate for the relatively weak binding affinity of G4 ligands. In addition, we employed buffers using potassium salts, rather than sodium, to mimic the intracellular ionic conditions and maintain the endogenous G4 landscape during the course of the experiment. For both G4 ligands we obtained high-quality maps revealing ca. 20,000 high confidence peaks for both G4 ligands over two biological and five technical replicates (FIG. 3B). In contrast, principal components analysis confirmed clear separation from biotin control experiments (FIG. 4C). Comparing the high confidence binding sites to published G4-seq data (OQS) (19), which comprises all sites that have the potential to form G4 structures in human genomic DNA, we found high overlap for both PDS (˜89%) and PhenDC3 (˜88%) (FIG. 3C and FIG. 4B). Next, we compared the small molecule binding sites to published maps of endogenous G4s, observed with BG4 CUT&Tag experiments. Validating our approach, we observed a strong overlap of high confidence binding sites as well signal correlation comparing antibody and G4 ligand maps (rₛ>0.7), and even higher correlation for PDS and PhenDC3 (rₛ>0.9) (FIG. 3D, E and 4B). These results are in accordance previous in vitro binding experiments finding that PDS, PhenDC3 and BG4 are relatively promiscuous G4 binders, with moderate G4 structure specificity (20). Nonetheless, the small molecules capture a different space of binding sites (5,690 consensus loci) that was not captured by BG4, which may be due different binding preference of the probes or accessibility of G4 structures (FIG. 3E). This data provides a strong validation of the mapped endogenous G4 landscape due to the orthogonality of antibody and small molecule probes. In addition, we observed similar trends mapping both G4 ligands in U-2 OS cells confirming the robustness of the Chemmap approach (FIG. 4F). Overall, high-resolution mapping of G4 ligands directly confirms drug-target engagement in chromatin and should also serve as a valuable tool to guide the development of improved G4 ligands and therapeutics.

To evaluate the general applicability of the approach to detect DNA-small molecules interactions, we studied a clinically approved drug doxorubicin believed to act by targeting DNA but has not been directly mapped to genomic DNA in cells. Doxorubicin belongs to the anthracycline class of antitumor antibiotics that can interact with DNA through an intercalation-type mechanism. The principal anticancer actions of anthracyclines are DNA intercalation, inhibition of topoisomerase II and formation of free radicals, which causes DNA structural changes, DNA damage and cellular cytotoxicity (1). Around 1 million cancer patients annually receive treatment with doxorubicin or its variants. However, despite many studies have been carried out on doxorubicin and its clinical use over last five decades, its mode of action is still not well-understood (21).

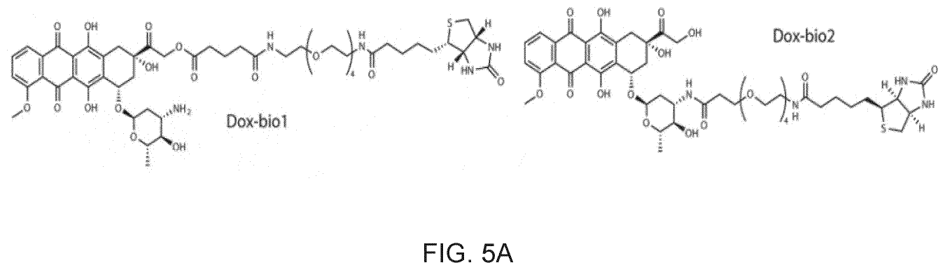

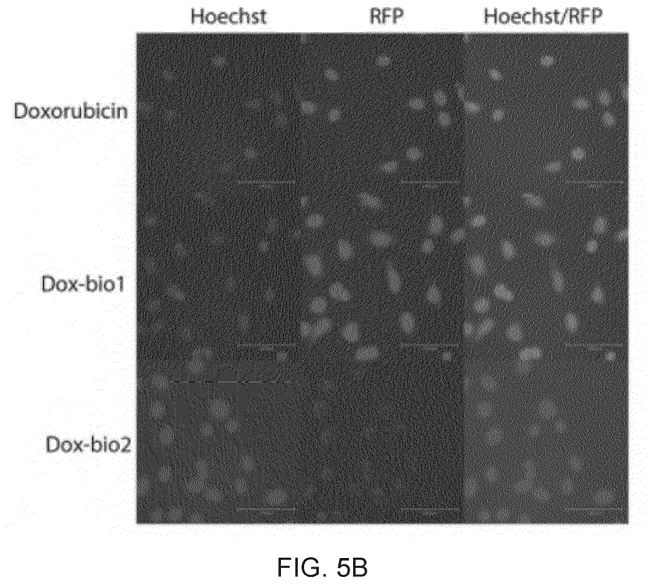

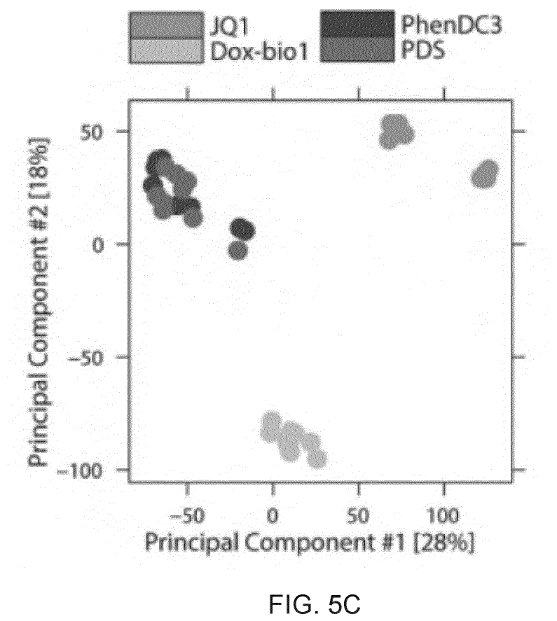

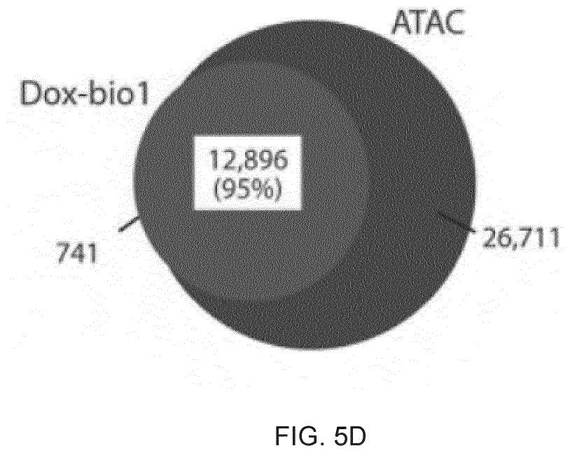

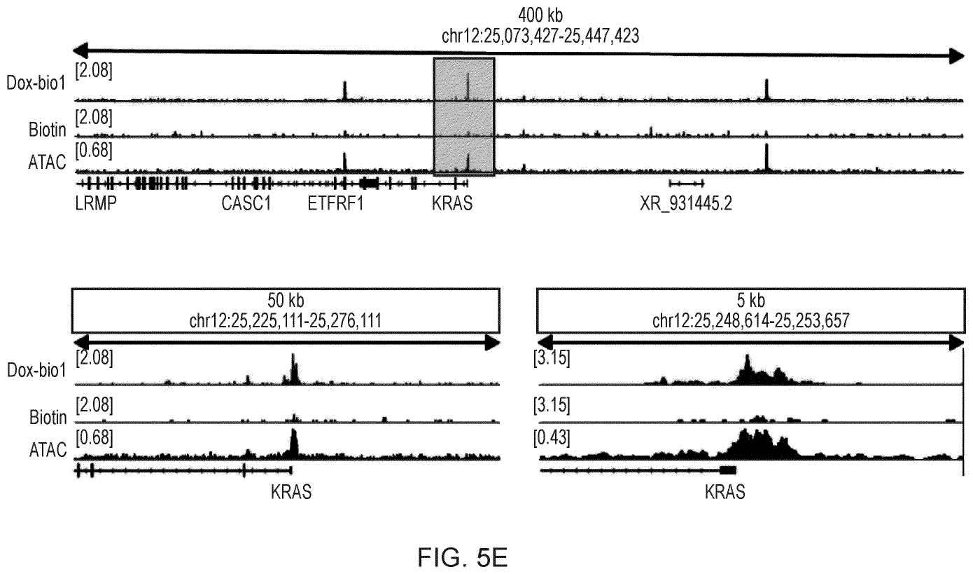

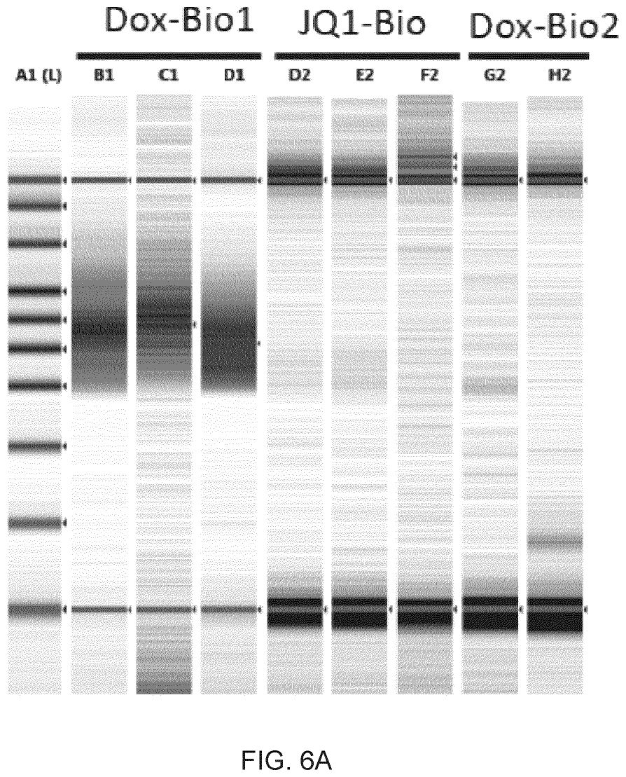

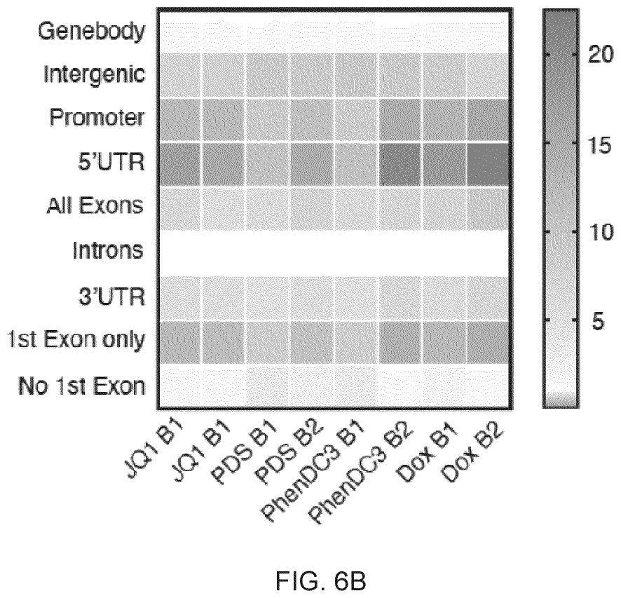

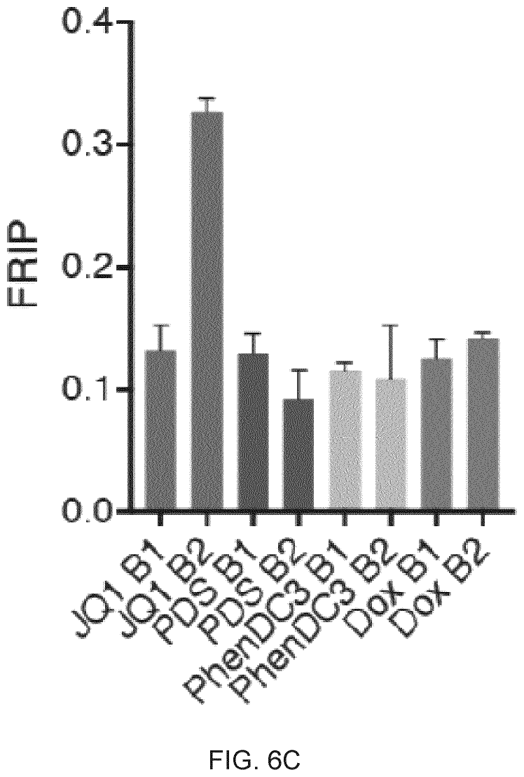

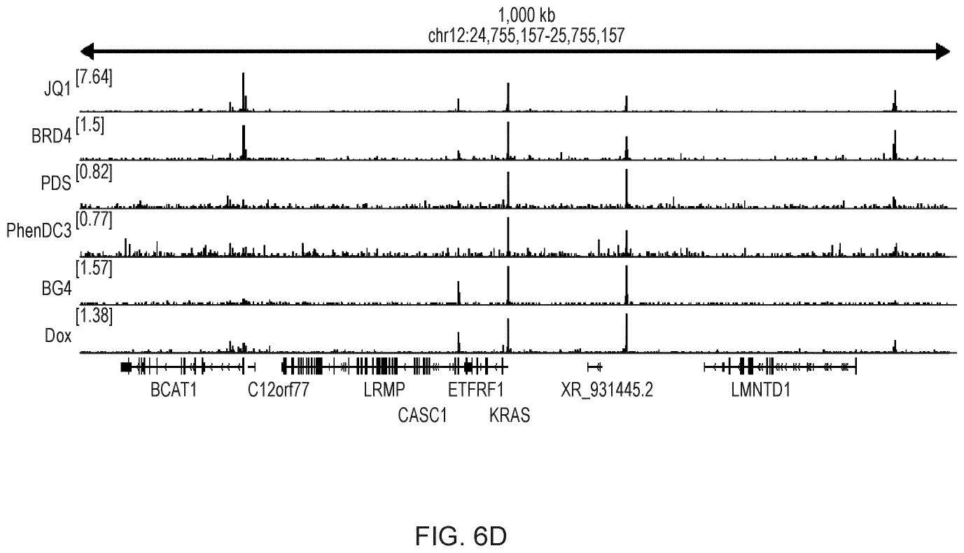

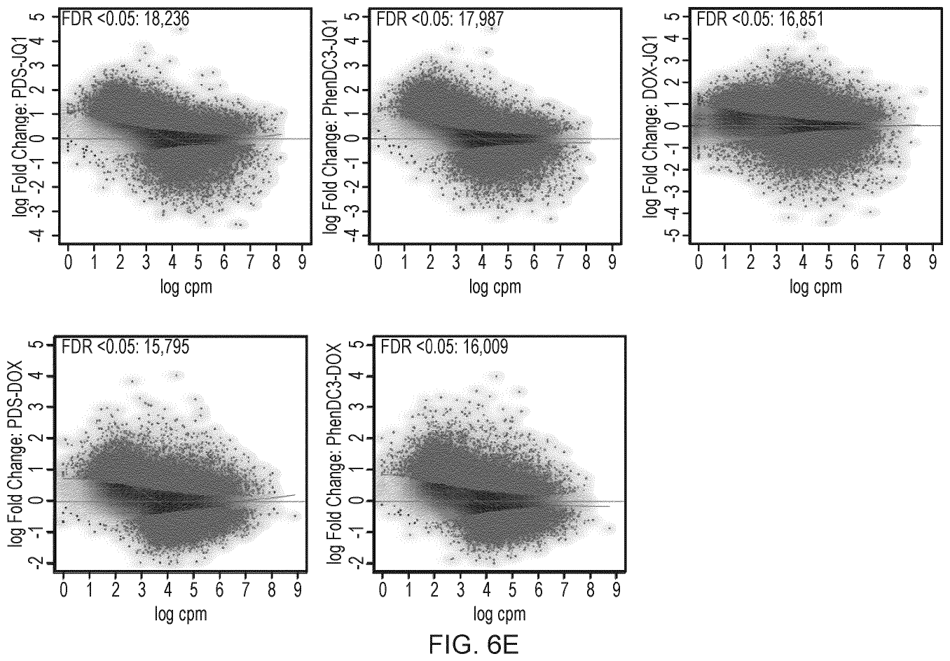

To use Chemmap approach, we first had to design an appropriately tagged doxorubicin. By following previous reports, two conjugation points were tested at 14-OH, 3′-NH2 resulting in biotinylated derivatives Dox-bio1 and Dox-bio2 (FIG. 5A). Next, we examined the cellular uptake of in U2OS cells using fluorescence microscopy utilizing the intrinsic fluorescence of doxorubicin (FIG. 5B). Notably, doxorubicin and Dox-bio1 were predominantly accumulated in the nuclei, while Dox-bio2 was mainly located in the cytoplasm. Similarly, we could not recover significant amounts of DNA in several attempts of Chemmap with Dox-bio2 in permeabilised K562 cells, highlighting the importance of 3′-NH2 for DNA binding events. In contrast, Chemmap with Dox-bio1 at different probe concentrations recovered substantial amounts of target DNA fragments from permeabilised K562 cells. Notably, 200 nM Dox-bio1 recovered up to 30-fold more material compared to other tested probes under same condition (FIG. 6B).

Depending on the probe concentration we observed around 14k high confidence Dox-bio1 binding sites. Interestingly, Dox-bio1 peaks that were predominantly (95%) located in open chromatin regions mapped by ATAC-seq (FIG. 5D). Given that we employed Dox-bio1 saturation conditions and that CUT&Tag type approaches readily map heterochromatin histone marks and protein binding (9), it is unlikely that this binding property is due to technical bias of the Chemmap approach. Our findings rather suggest that chromatin accessibility is a prerequisite for doxorubicin-DNA binding in cells (FIG. 5D) (22).

We then applied Chemmap to seek mechanistic evidence that epi-drugs can synergize chemotherapy via profiling the dynamics of doxorubicin binding. Histone deacetylases (HDAC) are key chromatin silencing modifiers and commonly dysregulated in cancers, making them an appealing therapeutic target for cancer treatment. Preclinic and clinical study demonstrated that HDAC inhibitor (HDACi) effectively sensitises cancer response to doxorubicin treatment, causing cell apoptosis and cell death (23, 24). We chose Tucidinostat (Chidamide), a selective HDAC inhibitor towards Class I HDAC1, HDAC2, HDAC3, as well as Class IIb HDAC10, which has been clinically approved for peripheral T-cell lymphoma (PTCL) and adult T-cell leukemia-lymphoma (ATLL) (25, 26) and combinatory treatment with doxorubicin in preclinic and clinical trials are on-going ((27), NCT04231448).

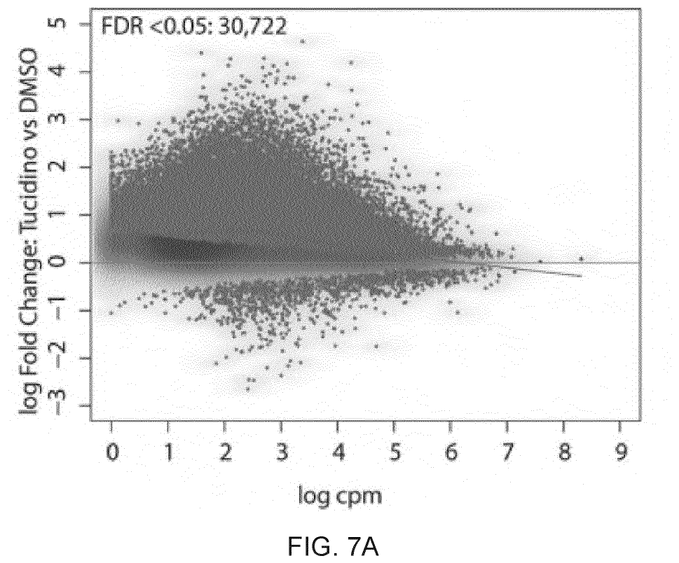

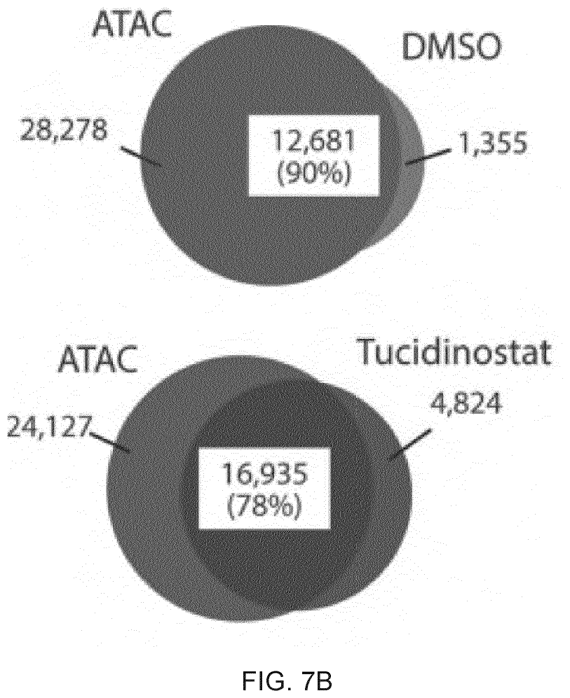

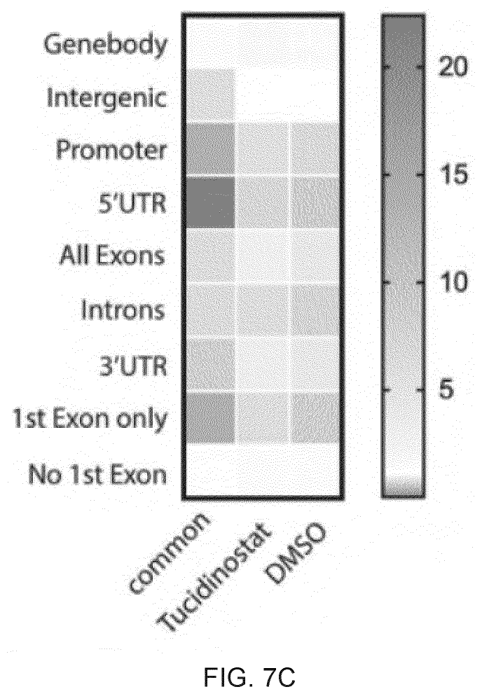

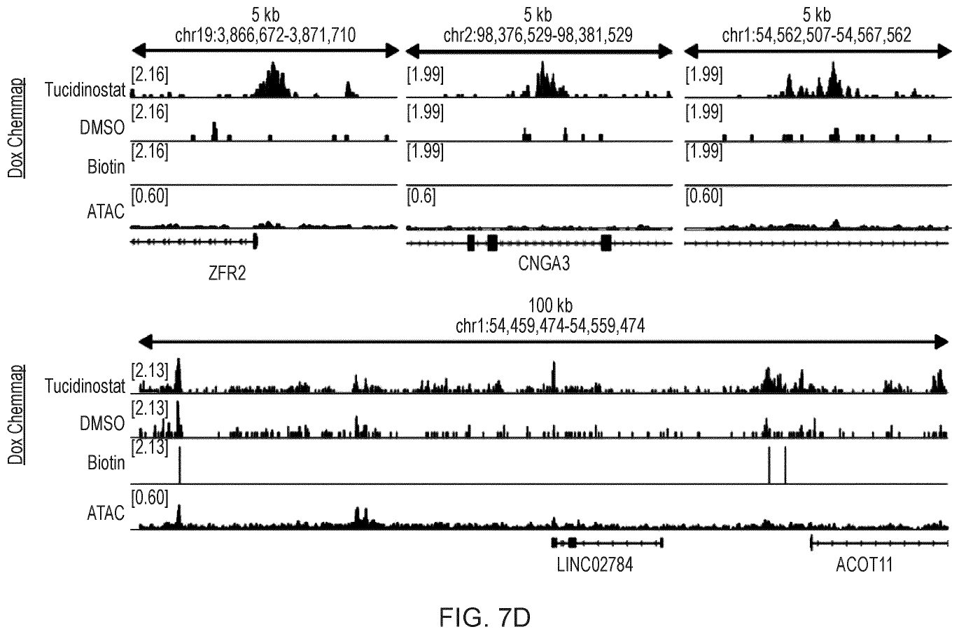

We recovered similar numbers of total high-confident peaks after 72 h treatment in K562 cells. Strikingly, differential binding analysis demonstrates that Tucidinostat treatment enhances the binding of doxorubicin and generates overwhelming levels of new binding sites (FIG. 7A and D). Also, HDACi pre-treatment expands the size of original binding loci. To investigate the genome distribution of new binding sites, Tucidinostat generates new binding sites which are outside of initial open chromatin region (42% in Tucidinostat compared with 95% in vehicle group), to suggest a significant chromatin remodelling progress (FIG. 4B). Overall, these results highlight that HDACi sensitizes cancer cells via expanding and newly establishing binding sites, to enhance the accessibility of Doxorubicin to the chromatin. Thus, Chemmap settles a mechanism-based approach to understand the rational for epi-drug combinatory therapeutics which would re-invigorate the field.

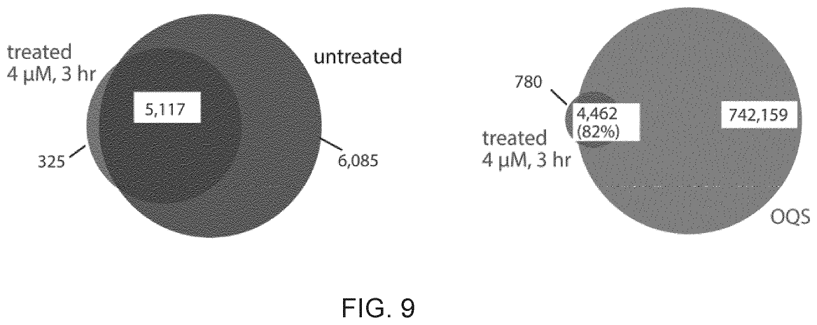

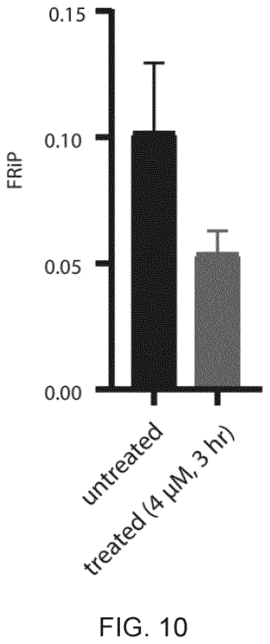

To confirm that Chemmap reflects small molecule binding sites in live cells, we treated K562 cells with unmodified PDS (4 μM for 3 hr) prior to PDS Chemmap (FIG. 9). We observed a drop in recovered DNA material and a considerable reduction in peak numbers with increased competitor concentrations and treatment times (˜6000 sites (60%) lost compared to untreated) (FIG. 10).

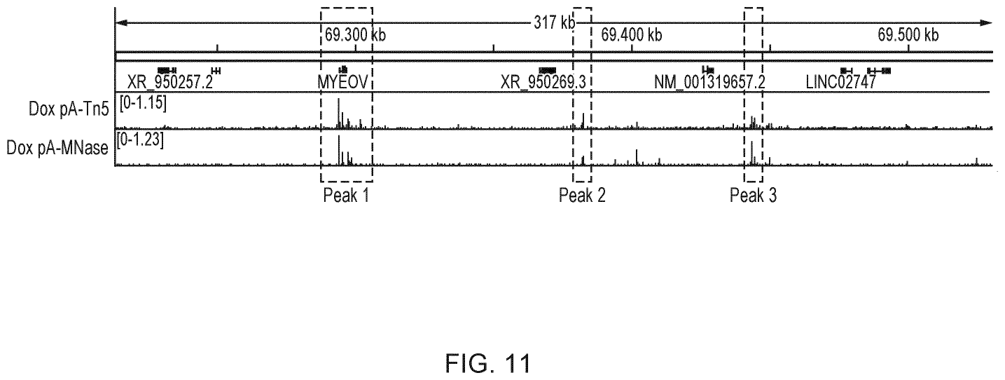

To confirm that Chemmap can be performed with different nucleases, we mapped the binding of biotinylated doxorubicin genome wide using both pA-Tn5 and pA-MNase. We found that doxorubicin mapped with pA-Tn5 and MNase show similar peak distribution (FIG. 11). This demonstrates that nucleases such as MNase can be used to map binding sites by Chemmap.

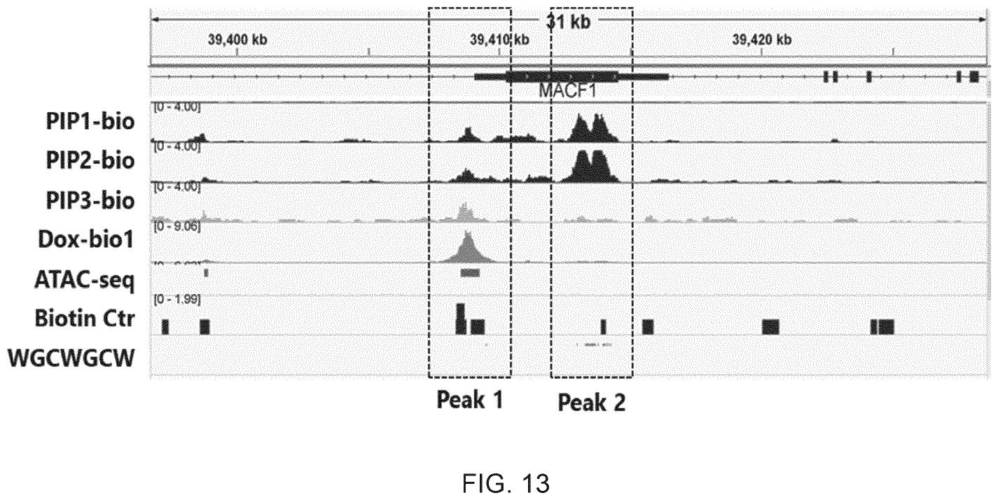

Pyrrole-imidazole polyamides are molecules which bind to the minor groove of DNA in a sequence specific manner. By adjusting the composition of the PIP, it is possible to modify the target DNA sequence that is recognised. Three biotinylated PIPs (FIG. 12) were used that bind to dsDNA with the following sequence specificity: WGCWGCW (PIP1-bio), WGCWGCW (PIP2-bio), and WWCWWWGW (PIP3-bio) (W stands for A or T). Following the Chemmap protocol and data analysis, PIP1-bio and PIP2-bio showed enriched binding at genomic loci with the DNA sequence WGCWGCW (FIG. 13).

We have demonstrated Chemmap as a robust, convenient, and unequivocal approach for mapping the interaction sites of small molecules in genomic DNA and chromatin protein in the cellular nucleus. The approach has generated insights on probe molecules and a clinically used drug that were previously not possible, and we demonstrate its general utility by mapping the interaction sites of chromatin protein inhibitor, two molecules that interact selectively with G-quadruplex structures and lastly a known DNA interactive compound doxorubicin that has been used for many years as an anticancer drug. Significantly, Chemmap helps to reveal the binding specificity of small molecules, to cross validate the fidelity of drug targets (G4 structure for instance), and to understand the mechanistic rational of combination treatment for diseases.

### REFERENCES

1. R. B. Silverman, M. W. Holladay, The organic chemistry of drug design and drug action. (Elsevier/AP, Academic Press, is an imprint of Elsevier, Amsterdam; Boston, ed. Third edition/, 2014), pp. xviii, 517 pages.
2. M. Schenone, V. Dančík, B. K. Wagner, P. A. Clemons, Target identification and mechanism of action in chemical biology and drug discovery. Nat. Chem. Biol. 9, 232-240 (2013).
3. L. Anders et al., Genome-wide localization of small molecules. Nat. Biotechnol. 32, 92-96 (2014).
4. R. Rodriguez, K. M. Miller, Unravelling the genomic targets of small molecules using high-throughput sequencing. Nat. Rev. Genet. 15, 783-796 (2014).
5. D. S. Tyler et al., Click chemistry enables preclinical evaluation of targeted epigenetic therapies. Science 356, 1397-1401 (2017).
6. J. L. Meier, A. S. Yu, I. Korf, D. J. Segal, P. B. Dervan, Guiding the design of synthetic DNA-binding molecules with massively parallel sequencing. J. Am. Chem. Soc. 134, 17814-17822 (2012).
7. C. Anandhakumar et al., Next-Generation Sequencing Studies Guide the Design of Pyrrole-Imidazole Polyamides with Improved Binding Specificity by the Addition of beta-Alanine. Chembiochem 15, 2647-2651 (2014).
8. E. G. S., B. Devesh, E. Asuka, A. A. Z., Mapping Polyamide—DNA Interactions in Human Cells Reveals a New Design Strategy for Effective Targeting of Genomic Sites. Angew. Chem. Int. Ed. Engl. 53, 10124-10128 (2014).
9. H. S. Kaya-Okur et al., CUT&Tag for efficient epigenomic profiling of small samples and single cells. Nat. Commun. 10, 1930 (2019).
10. P. Filippakopoulos et al., Selective inhibition of BET bromodomains. Nature 468, 1067-1073 (2010).
11. R. Rodriguez et al., A Novel Small Molecule That Alters Shelterin Integrity and Triggers a DNA-Damage Response at Telomeres. J. Am. Chem. Soc. 130, 15758-15759 (2008).
12. A. De Cian, E. Delemos, J. L. Mergny, M. P. Teulade-Fichou, D. Monchaud, Highly efficient G-quadruplex recognition by bisquinolinium compounds. J. Am. Chem. Soc. 129, 1856-1857 (2007).
13. S. Balasubramanian, L. H. Hurley, S. Neidle, Targeting G-quadruplexes in gene promoters: a novel anticancer strategy? Nat. Rev. Drug Discov. 10, 261-275 (2011).
14. R. Hänsel-Hertsch et al., G-quadruplex structures mark human regulatory chromatin. Nat. Genet. 48, 1267 (2016).
15. W. J. Chung, B. Heddi, F. Hamon, M.-P. Teulade-Fichou, A. T. Phan, Solution Structure of a G-quadruplex Bound to the Bisquinolinium Compound Phen-DC3. Angew. Chem. Int. Ed. 53, 999-1002 (2014).
16. S. Müller, S. Kumari, R. Rodriguez, S. Balasubramanian, Small-molecule-mediated G-quadruplex isolation from human cells. Nature Chem. 2, 1095-1098 (2010).
17. R. Rodriguez et al., Small-molecule-induced DNA damage identifies alternative DNA structures in human genes. Nat. Chem. Biol. 8, 301-310 (2012).
18. J. Lefebvre, C. Guetta, F. Poyer, F. Mahuteau-Betzer, M.-P. Teulade-Fichou, Copper-Alkyne Complexation Responsible for the Nucleolar Localization of Quadruplex Nucleic Acid Drugs Labeled by Click Reactions. Angew. Chem. Int. Ed. 56, 11365-11369 (2017).
19. V. S. Chambers et al., High-throughput sequencing of DNA G-quadruplex structures in the human genome. Nat. Biotechnol. 33, 877 (2015).
20. J. Spiegel, S. Adhikari, S. Balasubramanian, The Structure and Function of DNA G-Quadruplexes. Trends Chem. 2, 123-136 (2020).
21. X. Qiao et al., Uncoupling DNA damage from chromatin damage to detoxify doxorubicin. Proc. Natl. Acad. Sci. U.S.A. 117, 15182-15192 (2020).
22. J. Shen et al., Promoter G-quadruplex folding precedes transcription and is controlled by chromatin. Genome Biol. 22, 143 (2021).
23. P. N. Munster et al., Phase I trial of vorinostat and doxorubicin in solid tumours: histone deacetylase 2 expression as a predictive marker. Br. J. Cancer 101, 1044-1050 (2009).
24. K. Vu et al., Romidepsin Plus Liposomal Doxorubicin Is Safe and Effective in Patients with Relapsed or Refractory T-Cell Lymphoma: Results of a Phase I Dose-Escalation Study. Clin. Cancer Res. 26, 1000-1008 (2020).
25. D.-S. Pan et al., Discovery of an orally active subtype-selective HDAC inhibitor, chidamide, as an epigenetic modulator for cancer treatment. MedChemComm 5, 1789-1796 (2014).
26. Y. Shi et al., Results from a multicenter, open-label, pivotal phase II study of chidamide in relapsed or refractory peripheral T-cell lymphoma. Ann. Oncol. 26, 1766-1771 (2015).
27. M.-C. Zhang et al., Clinical efficacy and molecular biomarkers in a phase II study of tucidinostat plus R-CHOP in elderly patients with newly diagnosed diffuse large B-cell lymphoma. Clin. Epigenetics 12, 160 (2020).

1. A method of mapping the locations of one or more binding sites of a test compound within a nucleic acid comprising;
(i) contacting the nucleic acid with a tagged test compound comprising a test compound covalently linked to a tag, wherein the tagged test compound binds to the nucleic acid or to protein associated with the nucleic acid at one or more locations within the nucleic acid,
(ii) contacting the tagged test compound with a first binding member that specifically binds to the tag, such that the first binding member binds to the tagged test compound,
(iii) contacting the nucleic acid with a second binding member that specifically binds to the first binding member and is attached to an activatable nuclease, such that the second binding member binds to first binding member that is bound to the tagged test compound at the one or more binding sites,
(iv) activating the nuclease, such that the nuclease cleaves the nucleic acid at the one or more binding sites to generate fragments, and;
(v) determining the sequence of the generated fragments.

2. A method according to claim 1 wherein the sequences of the nucleic acid fragments are indicative of the locations of the one or more binding sites of the test compound within the nucleic acid.

3. A method according to claim 1 or claim 2 wherein the test compound binds to the nucleic acid at one or more locations within the nucleic acid.

4. A method according to claim 1 or claim 2 wherein the test compound binds to protein associated with the nucleic acid at one or more locations within the nucleic acid.

5. A method according to any one of the preceding claims wherein the test compound binds covalently to the nucleic acid or to protein associated with the nucleic acid.

6. A method according to any one of claims 1 to 4 wherein the test compound binds non-covalently to the nucleic acid or to protein associated with the nucleic acid.

7. A method according to any one of the preceding claims wherein the test compound is a small organic molecule of less than 5 KDa.

8. A method according to any one of the preceding claims wherein the tag is biotin

9. A method according to any one of the preceding claims wherein the nuclease is fused to an immunoglobulin binding moiety in a fusion protein, said fusion protein being non-covalently bound to the second binding member through the immunoglobulin binding moiety.

10. A method according to any one of the preceding claims wherein the activatable nuclease is micrococcal nuclease.

11. A method according to any one of the preceding claims wherein the activatable nuclease is a transposase.

12. A method according to claim 11 wherein the transposase is Tn5.

13. A method according to any one of claims 1 to 12 wherein steps (i) and (ii) are performed at the same time.

14. A method according to claim 13 wherein the method comprises contacting the nucleic acid with a complex that comprises the tagged test compound and the first binding member.

15. A method according to any one of claims 1 to 12 wherein steps (i) and (ii) are performed sequentially

16. A method according to any one of the preceding claims wherein the nucleic acid is in a eukaryotic nucleus or extract thereof.

17. A method according to any one of claims 1 to 15 wherein the nucleic acid is within a cell or cell extract.

18. A method according to claim 17 wherein the cell is a prokaryotic cell.

19. A method according to claim 17 wherein the cell is a eukaryotic cell.

20. A method according to any one of claims 17 to 19 wherein step (i) comprises culturing a viable cell in the presence of the tagged test compound.

21. A method according to claim 20 wherein the method further comprises permeabilising the cell before step (ii).

22. A method according to any one of the preceding claims wherein the nucleic acid is RNA.

23. A method according to claim 22 wherein the RNA is a cell transcriptome or fraction thereof.

24. A method according to any one of claims 1 to 21 wherein the nucleic acid is DNA.

25. A method according to claim 24 wherein the DNA is a cell genome or fragment thereof.

26. A method according to any one of claims 1 to 25 wherein the first binding member is an antibody.

27. A method according to any one of claims 1 to 26 wherein the second binding member is an antibody.

28. A method according to any one of claims 1 to 27 wherein the sequence of the generated fragments is determined by sequencing the fragments

29. A method according to any one of the preceding claims comprising generating a set of sequence reads of the nucleic acid fragments

30. A method according to claim 29 comprising mapping the sequence reads in the population to one or more locations in a reference genome.

31. A method according to any one of claims 1 to 27 wherein the sequence of the generated fragments is determined by amplifying the fragments.

32. A method according to claim 31 wherein the fragments are amplified using a set of primers specific for a nucleic acid sequence comprising a binding site of the test compound.

33. A method according to any one of the preceding claims comprising mapping the locations of one or more binding sites of a test compound within a first nucleic acid and a second nucleic acid and identifying the locations of one or more binding sites that are present in the first nucleic acid and not in the second nucleic acid or present in the second nucleic acid and not in the first nucleic acid.

34. A method according to claim 33 wherein the first nucleic acid is in a cell or an extract of a cell that has been subjected to a treatment and the second nucleic acid is in a cell or an extract of a cell that has not been subjected to the treatment.

35. A method according to claim 34 wherein the treatment is selected from exposure to one or more compounds; exposure to light or irradiation; or exposure to cell culture conditions.

36. A method according to any one of claims 1 to 27 wherein the sequence of the generated fragments is determined by amplifying the fragments.

37. A method according to claim 32 wherein the fragments are amplified using a set of primers specific for a nucleic acid sequence comprising a binding site of the test compound.

38. A method according to any one of the preceding claims wherein the nucleic acid is contacted with
(i) a population of tagged test compounds, each tagged test compound in the population comprising a test compound covalently linked to a tag and binding to the nucleic acid or to protein associated with the nucleic acid at one or more sites within the nucleic acid,
(ii) a population of primary binding members, each primary binding member in the population specifically binding to a different tagged test compound in the population, and
(iii) a population of secondary binding members attached to activatable nucleases, each secondary binding member in the population specifically binding to a different primary binding member bound to a tagged test compound at a binding site.

39. A method according to any one of claims 1 to 37 wherein step (i) further comprises contacting the nucleic acid with an untagged second test compound, optionally wherein the untagged second test compound binds to the nucleic acid or to protein associated with the nucleic acid at one or more locations within the nucleic acid.

40. A method according to claim 39 comprising determining the effect of the presence of the untagged second test compound on the sequences of the fragments generated by the tagged test compound.

41. A kit for mapping the locations of one or more binding sites of a test compound within a nucleic acid comprising;
a tag covalently linked or linkable to a test compound
a first binding member that specifically binds to the tag,
a second binding member that specifically binds to the first binding member,
a nuclease that is attached or attachable to the second binding member.

42. A kit according to claim 41 for use in method according to any one of claims 1 to 41.
